(19) United States
(12) Patent Application Publication
(10) Pub. No.: US 2024/0036060 A1
(43) Pub. Date: Feb. 1, 2024

(54) OPTIMAL COMBINATION OF EARLY BIOMARKERS FOR INFECTION AND SEPSIS DIAGNOSIS IN EMERGENCY DEPARTMENT

(71) Applicants: INSTITUT PASTEUR, PARIS (FR); ASSISTANCE PUBLIQUE - HOPITAUX DE PARIS, PARIS (FR); SORBONNE UNIVERSITE, PARIS (FR); BIO-RAD EUROPE GMBH, BASEL (CH)

(72) Inventors: Jean-Marc CAVAILLON, CHAVILLE (FR); Laetitia VELLY, PARIS (FR); Pierre HAUSFATER, PARIS (FR); Catherine FITTING, CHAVILLE (FR); Stevenn VOLANT, CLAMART (FR); Florian SALIPANTE, NIMES (FR); Lionel VALERA, GRABELS (FR); Jeannette FAREH, SAUSSINES (FR)

(21) Appl. No.: 18/247,718

(22) PCT Filed: Oct. 11, 2021
(86) PCT No.: PCT/IB2021/000715

§ 371 Date: Apr. 3, 2023

Related U.S. Application Data
(60) Provisional application No. 63/089,635, filed on Oct. 9, 2020.

(51) Int. Cl. G01N 33/68

(52) U.S. Cl. CPC G01N 33/6893; G01N 2800/26

(57) ABSTRACT

The present invention allows a rapid and early diagnosis of bacterial or viral infection. Such a diagnosis is highly desired among patients admitted in emergency department to allow the initiation of the appropriate treatment. Although no biomarker alone can offer an appropriate diagnosis with sufficient sensitivity and specificity, the present invention defines optimal combinations of biomarkers allowing the diagnosis of infection.

### BACKGROUND OF THE INVENTION

The recent update in sepsis definitions have reinforced sepsis as a life-threatening organ dysfunction caused by a dysregulated host response to infection [1]. Because clinical signs at patient's presentation are often non-specific, sepsis biomarkers have been intensively investigated with the objective of improving sepsis identification and promoting early therapeutic bundles implementation [2]. In this very active field of research, the majority of studies have been conducted in critical care setting. However, most of sepsis patients attend hospitals through the emergency departments (ED) [3]. ED have therefore a pivotal role for the early identification of sepsis but also infection, which is the pre-requisite for sepsis suspicion. An inflammatory profile is observed in many ED clinical situations and the challenge is to identify among them, those patients who really have infection.

Due to complex and multimodal pathophysiology pathways, currently no individual biomarker of infection and/or sepsis is sufficiently discriminant to allow proper diagnosis [4]. The aim of the “Biomarqueurs d'Identification Précoce du Sepsis aux urgences” (BIPS) study was to measure in patients suspected of having infection or sepsis in the emergency department, a panel of biomarkers (covering several distinct pathophysiological pathways) and to identify the best combination that could provide high specificity and sensitivity for bacterial infection, viral infection and sepsis diagnosis. The originality of the BIPS study is that compared with intensive care units, the patients investigated for suspected sepsis in the ED are seen earlier in their medical history and can provide blood samples before any therapeutic intervention (such as fluid resuscitation, antibiotics, vasopressors) potentially interfering with several biomarkers of interest.

Despite a large number of biomarkers has been reported to allow the diagnosis of infection or sepsis, none had sufficient specificity or sensitivity to be routinely employed in clinical practice [4]. When the authors unanimously concluded that there was no magic maker, there was a hope that a combination of biomarkers could be more appropriate [2]. For example, the association of CRP (C-reactive protein) and neutrophil CD64 (cluster of differentiation 64) [31], or that of PCT (procalcitonin), soluble TREM-1 and neutrophil CD64 were shown promising. However, a large multicenter study including 29 plasma biomarkers, 14 cell surface biomarkers and 10 mRNA failed to find any combination useful for the diagnosis of sepsis-2 among ICU (intensive care unit) patients [33]. Also, a study analyzing numerous cell surface biomarkers concluded that no combination had clinically relevant predictive validity for the diagnosis of sepsis among patients with suspected acute infection [35]. Indeed, most studies carried out for the diagnosis of sepsis have been performed in intensive care units, among highly inflammatory patients. For these patients, the occurrence of an infection could be blinded within a storm of inflammatory biomarkers and highly altered expression of cell-surface makers. In this context, the new definition of “sepsis-3” is of limited interest for patients admitted in ED, since it is associated with organ failure of easy diagnosis. The presence of an infection remains in fact the most important challenge to address for these patients.

### DETAILED DESCRIPTION OF THE INVENTION

Very few studies have reported combinations allowing to decipher between bacterial and viral infection. Oved and colleagues have reported the interest to measure three plasma markers (CRP, TRAIL (TNF-related apoptosis-inducing ligand), and CXCL10). While the combination of gene expressions has led to numerous positive investigations for the diagnosis of bacterial infection [46; 45], only one study revealed that a set of seven genes could allow a robust discrimination between bacterial and viral infection [44].

A monocentric prospective investigation has been performed on 308 patients. Sixteen different biomarkers measured in plasma, and eleven biomarkers measured on monocytes, neutrophils, B and T-lymphocytes were included. In addition, a bacterial biomarker (endotoxin linked to leukocytes) was investigated. The final analysis has been made on 291 patients of whom 148 had bacterial infection, and 47 had viral infection. Among the patients with bacterial infection, 70 had sepsis according to the 2002 definition (hereafter “sepsis-2”) and 16 had sepsis according to the 2016 definition (hereafter “sepsis-3”).

Measuring different biomarkers of patients at admission in the emergency department, four different combinations of plasma and cell surface biomarkers were identified as being strongly associated with a very good diagnosis value of bacterial and viral infection, as well as of sepsis-2 and -3. Interestingly, only a limited amount of markers was sufficient to end up with a diagnosis having both a high sensitivity and a high specificity.

The present inventors have identified a first combination of three markers which allows the diagnosis of bacterial infection with high accuracy. This combination includes to measure the level of HLA-DR (human leukocyte antigen DR) expressed by monocytes, the level of MerTk (Myeloid-epithelial-reproductive tyrosine kinase) expressed by neutrophils and, as a third biomarker, the level of plasmatic Metalloproteinase-8 (MMP8). Interestingly, this combination allowed an area under the curve (AUC) of 0.934. More precisely, the association of HLA-DR (% on CD14+ monocytes), and that of MerTK (% on CD66+ neutrophils) ended to an AUC of 0.921 [0.89-0.95]. The addition of a third biomarker (MMP8) further improved the AUC=0.934 [0.91-0.96] with a sensitivity of 0.865 and a specificity of 0.902.

Among the patients who had been identified not to be affected by a bacterial infection, the inventors furthermore identified a combination of three other markers which allowed to diagnose those suffering from a viral infection. This second combination includes measuring the level of CD64 and CD24 expression on neutrophils and the level of CX3CR1 expression on monocytes. Interestingly, this second combination ended to an AUC=0.97. More precisely, the association of CD64 and CX3CR1 (ended to an AUC of 0.955 [0.92-0.99]. The addition of a third biomarker on neutrophil (mean fluorescence intensity (MFI) of CD24) ended to an AUC of 0.97 [0.95-0.99]. The sensitivity of these three biomarkers was 0.936 and its specificity 0.875.

Because the present study was designed before the sepsis-3 definition became available, a first analysis was done to identify the best combination to define sepsis-2 patients. Seventy patients entered in the sepsis-2 definition. The best combination to identify patients suffering from sepsis-2 was obtained by measuring the HLA-DR expression on monocytes and the plasmatic procalcitonine (PCT) and IL-6 biomarkers (AUC=0.891).

Regarding the classification of patients as sepsis-3 (n=16), the inventors herein show that the best combination can be obtained by measuring the HLA-DR expression on monocytes and the plasmatic hyaluronan (HA) and creatinine (AUC=0.97). Thus, the best combination was HLA-DR together with hyaluronan and creatinine.

This study is one of the very rare to allow the diagnosis of bacterial infection and that of viral infection in ED. Because new technologies are rapidly developing, such as the measurement of the expression of cell surface biomarkers by microfluidic [50; 51], and needle shaped microelectrode for electrochemical detection of biomarker in real time [50], a combination of cell surface biomarkers and plasma biomarkers should not be a problem to achieve diagnosis at bedside.

### Methods for Blood Analysis

In a first aspect, the invention encompasses methods for blood analysis. In one embodiment, the method comprises providing a blood sample from a patient prior to the administration of any fluid resuscitation, antibiotics, or vasopressors to the patient. Within the context of this invention, “prior to the administration of any fluid resuscitation, antibiotics, or vasopressors to the patient” means that the patient has not received any fluid resuscitation, antibiotics, or vasopressors within one month prior to providing the blood sample.

In some embodiments, the method comprises isolating cells from the blood sample. Preferably, the method comprises isolating monocytes and/or isolating neutrophils from the blood sample as explained below. In some embodiments, the method comprises isolating plasma from the blood sample, with conventional means, such as centrifugation.

The blood and plasma samples are to be kept at +4° C. or −80° C. until cell surface markers assessment or biomarkers measurements. The delays between blood draw and flow cytometry analysis, plasma collection and freezing are preferably of 2 to 8 hours.

In some embodiments, the method comprises measuring the level of HLA-DR (human leukocyte antigen DR) expression on the monocytes. HLA-DR expression on the monocytes can be measured by routine techniques in the art, for example, as detailed in the definitions below.

In some embodiments, the method comprises measuring the level of MerTk (Myeloid-epithelial-reproductive tyrosine kinase) expression on the neutrophils. The level of MerTk on the neutrophils can be measured by routine techniques in the art, for example, as detailed in the definitions below.

In some embodiments, the method comprises measuring the level of Metalloproteinase-8 (MMP8) in the plasma. The level of Metalloproteinase-8 (MMP8) in the plasma can be measured by routine techniques in the art, for example, as detailed in the definitions below.

In a preferred embodiment, the method comprises measuring the level of HLA-DR (human leukocyte antigen DR) expression on the monocytes, measuring the level of MerTk (Myeloid-epithelial-reproductive tyrosine kinase) on the neutrophils, and measuring the level of Metalloproteinase-8 (MMP8) in the plasma.

In some embodiments, the method comprises measuring the level of CD64 expression on the neutrophils. The level of CD64 on the neutrophils can be measured by routine techniques in the art, for example, as detailed in the definitions below.

In some embodiments, the method comprises measuring the level of CD24 expression on the neutrophils. The level of CD24 on the neutrophils can be measured by routine techniques in the art, for example, as detailed in the definitions below.

In some embodiments, the method comprises measuring the level of CX3CR1 expression on the monocytes. The level of CX3CR1 on the monocytes can be measured by routine techniques in the art, for example, as detailed in the definitions below.

In a preferred embodiment, the method comprises measuring the level of CD64 expression on the neutrophils, measuring the level of CD24 on the neutrophils, and measuring the level of CX3CR1 on the monocytes.

In some embodiments, the method comprises measuring the level of procalcitonin (PCT) in the plasma. The level of procalcitonin in the plasma can be measured by routine techniques in the art, for example, as detailed in the definitions below.

In some embodiments, the method comprises measuring the level of IL-6 in the plasma. The level of IL-6 in the plasma can be measured by routine techniques in the art, for example, as detailed in the definitions below.

In a preferred embodiment, the method comprises measuring the level of HLA-DR (human leukocyte antigen DR) expression on the monocytes, measuring the level of procalcitonin in the plasma, and measuring the level of IL-6 in the plasma.

In some embodiments, the method comprises measuring the level of hyaluronan in the plasma. The level of hyaluronan in the plasma can be measured by routine techniques in the art, for example, as detailed in the definitions below.

In some embodiments, the method comprises measuring the level of creatinine in the plasma. The level of creatinine in the plasma can be measured by routine techniques in the art, for example, as detailed in the definitions below.

In a preferred embodiment, the method comprises measuring the level of HLA-DR (human leukocyte antigen DR) expression on the monocytes, measuring the level of hyaluronan in the plasma, and measuring the level of creatinine in the plasma.

In other preferred embodiment, the method further comprises measuring the level of CD169 expression on monocytes, so as to discriminate between bacterial and viral infections, as proposed in Bourgoin et al, 2020 ([48]).

In some embodiments, the patient has a bacterial infection. Apart from the biomarker signature of the invention, a bacterial infection can be measured by routine techniques in the art, for example, by using selective culturing detect specific bacterial growth properties, ELISA to detect specific bacterial antigens, or DNA detection techniques (such as PCR) to detect specific bacterial DNA. In some embodiments, the patient does not have a bacterial infection.

In some embodiments, the patient has a viral infection. Apart from the biomarker signature of the invention, a viral infection can be measured by routine techniques in the art, for example, by using ELISA to detect specific viral antigens, or DNA/RNA detection techniques (such as PCR) to detect specific viral nucleic acids. In some embodiments, the patient does not have a viral infection.

In some embodiments, the patient has sepsis-2. In some embodiments, the patient has sepsis-3. These classifications are well-known to the skilled artisan ([49]). In some embodiments, the patient does not suffer from sepsis.

In particularly preferred embodiments, one or more of the cell surface markers are measured with a microfluidic biochip comprising antibodies against the cell surface marker(s) or other technical means, as disclosed in the definitions below.

In preferred embodiments, the combinations of biomarkers to be detected are:

### Bacterial Infection

A combination of HLA-DR+MerTk
A combination of HLA-DR+MerTk+IL-6
A combination of HLA-DR+MerTk+White Blood Cells (WBC)
A combination of HLA-DR+MMP8
A combination of HLA-DR+White Blood Cells (WBC)
A combination of MMP8+MerTk
The best combination being HLA-DR+MerTk+MMP8

### Viral Infection

A combination of CD64+CX3CR1
A combination of CD64+IP10
A combination of CD64+CD24
A combination of CX3CR1+CD24
The best combination being CD64+CD24+CX3CR1

### Sepsis-2

A combination of HLA-DR+sCD14
A combination of IL-6+PCT+CRP (C-reactive protein)
A combination of HLA-DR+IL-6
A combination of HLA-DR+CRP (C-reactive protein)
A combination of HLA-DR+PCT
A combination of HLA-DR+MMP8
A combination of HLA-DR+MerTk
A combination of PCT+IL-6
The best combination being: HLA-DR+PCT+IL-6

### Sepsis-3

A combination of hyaluronan+creatinine
A combination of hyaluronan+HLA-DR
A combination of creatinine+HLA-DR
The best combination being HLA-DR+hyaluronan+creatinine

In the methods of the invention, expression of cell surface receptors and/or cell surface antigens (on any cells, including blood and/or plasma cells, such as monocytes, neutrophils) are preferably assessed (measured, or identified, or detected) using well known analytical technologies such as flow cytometry. It is in particular possible to use rapid flow-cytometry protocols such as those disclosed in [39] and [47], which are incorporated herein by reference. On another hand, the amount of plasmatic biomarkers can be assessed (measured, or identified, or detected) using well known analytical technologies such as ELISA or fluorescence immunoassays.

### Methods and Uses of Biomarkers for Diagnosis

In another aspect, the invention encompasses methods and uses of biomarkers for diagnosis. In particular, the invention encompasses the use of the above-defined biomarkers for diagnosing bacterial infection, viral infection, sepsis-2 or sepsis-3 in patients attending hospitals, and more specifically their emergency departments.

In a preferred embodiment, said method or use comprises measuring a decreased level of HLA-DR (human leukocyte antigen DR) expression on the monocytes, measuring an increased level of MerTk (Myeloid-epithelial-reproductive tyrosine kinase) expression on the neutrophils, and measuring an increased level of Metalloproteinase-8 (MMP8) in the plasma. These tendencies are diagnostic for a bacterial infection. In other words, it can be said that the patient suffers from a bacterial infection if he/she displays a decreased level of HLA-DR (human leukocyte antigen DR) expression on the monocytes, an increased level of MerTk (Myeloid-epithelial-reproductive tyrosine kinase) expression on the neutrophils, and an increased level of Metalloproteinase-8 (MMP8) in the plasma, as compared to reference levels. By contrast, it can not be concluded that the patient suffers from a bacterial infection if he/she displays an increased level of HLA-DR (human leukocyte antigen DR) expression on the monocytes, or a decreased level of MerTk (Myeloid-epithelial-reproductive tyrosine kinase) expression on the neutrophils, or a decreased level of Metalloproteinase-8 (MMP8) in the plasma, as compared to reference levels.

In a preferred embodiment, said method or use further comprises measuring a decreased level of CD64 expression on the neutrophils, measuring a decreased level of CD24 expression on the neutrophils, and measuring an increased level of CX3CR1 expression on the monocytes. These tendencies are diagnostic for a viral infection. In other words, for patients that do not suffer from a bacterial infection (as concluded by the above-mentioned method), it can be said that the patient suffers from a viral infection if he/she displays a decreased level of CD64 expression on the neutrophils, a decreased level of CD24 expression on the neutrophils, and an increased level of CX3CR1 expression on the monocytes as compared to reference levels. By contrast, it cannot be concluded that the patient suffers from a viral infection if he/she displays an increased level of CD64 expression on the neutrophils, or an increased level of CD24 expression on the neutrophils, or a decreased level of CX3CR1 expression on the monocytes, as compared to reference levels.

Alternatively, said method or use comprises measuring a decreased level of HLA-DR (human leukocyte antigen DR) expression on the monocytes, measuring an increased level of procalcitonin in the plasma, and measuring an increased level of IL-6 in the plasma. These tendencies are diagnostic for a sepsis-2. In other words, it can be said that the patient suffers from sepsis-2 if he/she displays a decreased level of HLA-DR (human leukocyte antigen DR) expression on the monocytes, an increased level of procalcitonin in the plasma, and an increased level of IL-6 in the plasma, as compared to reference levels. By contrast, it cannot be concluded that the patient suffers from sepsis-2 if he/she displays an increased level of HLA-DR (human leukocyte antigen DR) expression on the monocytes, or a decreased level of procalcitonin in the plasma, or a decreased level of IL-6 in the plasma, as compared to reference levels.

Alternatively, said method or use comprises measuring a decreased level of HLA-DR (human leukocyte antigen DR) expression on the monocytes, measuring a decreased level of hyaluronan in the plasma, and measuring an increased level of creatinine in the plasma. These tendencies are diagnostic for a sepsis-3. In other words, it can be said that the patient suffers from sepsis-3 if he/she displays a decreased level of HLA-DR (human leukocyte antigen DR) expression on the monocytes, a decreased level of hyaluronan in the plasma, and an increased level of creatinine in the plasma, as compared to reference levels. By contrast, it cannot be concluded that the patient t suffers from sepsis-3 if he/she displays an increased level of HLA-DR (human leukocyte antigen DR) expression on the monocytes, or an increased level of hyaluronan in the plasma, or a decreased level of creatinine in the plasma, as compared to reference levels.

In one aspect, the invention encompasses methods for diagnosis of a bacterial infection, said methods comprising:
a) providing a blood sample from a patient, preferably prior to the administration of any fluid resuscitation, antibiotics, or vasopressors to the patient,
b) measuring the level of HLA-DR (human leukocyte antigen DR) expression on the monocytes in said blood sample,
c) measuring the level of MerTk (Myeloid-epithelial-reproductive tyrosine kinase) expression on the neutrophils in said blood sample,
d) measuring the level of Metalloproteinase-8 (MMP8) in the plasma of said blood sample, and
e) diagnosing the patient with a bacterial infection, if a decreased level of HLA-DR expression, an increased level of MerTk expression, and an increased level of MMP8 are observed as compared to reference levels.

In another aspect, the invention encompasses methods for diagnosis of a viral infection, said methods comprising:
a) measuring the level of CD64 expression on neutrophils in the blood sample,
b) measuring the level of CD24 expression on neutrophils in the blood sample, and
c) measuring the level of CX3CR1 expression on monocytes in the blood sample, and
d) diagnosing the patient with a viral infection, if a decreased level of CD64 expression, a decreased level of CD24 expression, and an increased level of CX3CR1 expression are observed as compared to reference levels.

In another aspect, the invention encompasses methods for diagnosis of a bacterial or viral infection, said methods comprising:
a) providing a blood sample from a patient, preferably prior to the administration of any fluid resuscitation, antibiotics, or vasopressors to the patient,
b) measuring the level of HLA-DR (human leukocyte antigen DR) expression on the monocytes in said blood sample,
c) measuring the level of MerTk (Myeloid-epithelial-reproductive tyrosine kinase) expression on the neutrophils in said blood sample,
d) measuring the level of Metalloproteinase-8 (MMP8) in the plasma of said blood sample, and
e) diagnosing the patient with a bacterial infection, if a decreased level of HLA-DR expression, an increased level of MerTk expression, and an increased level of MMP8 are observed as compared to reference levels,
f) measuring the level of CD64 expression on neutrophils in the blood sample,
g) measuring the level of CD24 expression on neutrophils in the blood sample, and
h) measuring the level of CX3CR1 expression on monocytes in the blood sample, and
i) diagnosing the patient with a viral infection, if a decreased level of CD64 expression, a decreased level of CD24 expression, and an increased level of CX3CR1 expression are observed as compared to reference levels.

In these methods, the steps can be performed in any order, or simultaneously. Also, in this method, it is possible not to perform all the steps leading to the diagnostic of one type of infection, if one of the marker is not increased/decreased as expected herein. For example, if the HLA-DR expression is increased in the monocytes of the sample, then the subject does not suffer from bacterial infection, there is therefore no need to perform the two other measurements c) and d) and the steps f) or g) or h) can be performed rapidly.

Although there is no need to perform all the three steps in the methods of the invention if one marker is not increased/decreased as expected, it is always preferred to confirm the results by checking the other markers.

In other preferred embodiment, the method further comprises measuring the level of CD169 expression on monocytes, so as to discriminate between bacterial and viral infections, as proposed in [48].

These methods can be performed on patients arriving at the hospital without knowing anything about their infection status, or on patients that have been already diagnosed not to suffer from a bacterial infection with the biomarkers HLA-DR, MerTK and MMP8.

In another aspect, the invention encompasses methods for diagnosis of sepsis-2, said methods comprising:
a) providing a blood sample from a patient, preferably prior to the administration of any fluid resuscitation, antibiotics, or vasopressors to the patient,
b) measuring the level of HLA-DR (human leukocyte antigen DR) expression on the monocytes in the blood sample,
c) measuring the level of procalcitonin in the plasma of the blood sample,
d) measuring the level of IL-6 in the plasma of the blood sample, and
e) diagnosing the patient with sepsis-2, if a decreased level of HLA-DR expression, an increased level of procalcitonin, and an increased level of IL-6 are observed as compared to reference levels.

In another aspect, the invention encompasses methods for diagnosis of sepsis-3, said methods comprising:
a) providing a blood sample from a patient, preferably prior to the administration of any fluid resuscitation, antibiotics, or vasopressors to the patient,
b) measuring the level of HLA-DR (human leukocyte antigen DR) expression on the monocytes in the blood sample,
c) measuring the level of hyaluronan in the plasma of the blood sample,
d) measuring the level of creatinine in the plasma of the blood sample, and
e) diagnosing the patient with sepsis-3, if a decreased level of HLA-DR expression, a decreased level of hyaluronan, and an increased level of creatinine are observed as compared to reference levels.

These methods to diagnose sepsis can be performed on patients arriving at the hospital with a suspected infection, yet without knowing anything about the origin of their infection, or on patients that have been already diagnosed to suffer from a bacterial infection with the biomarkers HLA-DR, MerTK and MMP8, or from a viral infection with the biomarkers CD64, CD24, and CX3CR1 as proposed in the other methods of the invention.

In the methods of the invention, expression of cell surface receptors and/or cell surface antigens (on any cells, including blood and/or plasma cells, such as monocytes, neutrophils) are preferably assessed (measured, or identified, or detected) using well known analytical technologies such as flow cytometry. It is in particular possible to use rapid flow-cytometry protocols such as those disclosed in [39] and [47], which are incorporated herein by reference. On another hand, the amount of plasmatic biomarkers can be assessed (measured, or identified, or detected) using well known analytical technologies such as ELISA or fluorescence immunoassays.

### Kits of the Invention

The present invention furthermore encompasses kits that contain all the necessary technical means to implement the methods of the invention.

In particular, said means are those that can easily detect:
The level of HLA-DR expression at the cell surface: in a preferred embodiment, one can use specific antibodies that binds with high affinity to HLA-DR. These antibodies are commercially available.
The level of MerTK expression at the cell surface: in a preferred embodiment, one can use specific antibodies that binds with high affinity to MerTK. These antibodies are commercially available.
The level of CD64 expression at the cell surface: in a preferred embodiment, one can use specific antibodies that binds with high affinity to CD64. These antibodies are commercially available.
The level of CD24 expression at the cell surface: in a preferred embodiment, one can use specific antibodies that binds with high affinity to CD24. These antibodies are commercially available.
The level of CX3CR1 expression at the cell surface: in a preferred embodiment, one can use specific antibodies that binds with high affinity to CX3CR1. These antibodies are commercially available.
The level of circulating MMP8: in a preferred embodiment, one can use specific antibodies that binds with high affinity to MMP8. These antibodies are commercially available.
The level of circulating PCT: in a preferred embodiment, one can use specific antibodies that binds with high affinity to PCT. These antibodies are commercially available.
The level of circulating IL-6: in a preferred embodiment, one can use specific antibodies that binds with high affinity to IL-6. These antibodies are commercially available.
The level of circulating hyaluronan: in a preferred embodiment, one can use specific antibodies that binds with high affinity to hyaluronan. These antibodies are commercially available.
The level of circulating creatinine: in a preferred embodiment, one can use specific antibodies that binds with high affinity to creatinine. These antibodies are commercially available.

Advantageously, each of the antibodies (e.g., anti-HLA-DR, anti-CD24, anti-CD64, anti-CX3CR1 and other antibodies) is labelled with a specific fluorochrome or fluorescence agent (as used herein: “labelled antibody”), enabling the cytometer to identify the contaminant cells carrying the antigen recognized by said antibody, and thus the selection of the cells which do not carry the antigen. The fluorochromes which can be used are well known in the art. They include such fluorochromes as e.g., PE, APC, PE-Cy5, Alexa Fluor 647, PE-Cy-7, PerCP-Cy5.5, Alexa Fluor 488, Pacific Blue, FITC, AmCyan, APC-Cy7, PerCP, and APC-H7.

In a preferred embodiment, the kit of the invention contains or consists essentially in all the means to distinguish between a viral and a bacterial infection, that is namely antibodies that specifically detect HLA-DR, MerTK, and MMP8. Another preferred kit contains or consists essentially in antibodies that specifically detect CD64, CD24, and CX3CR1. Another preferred kit contains or consists essentially in antibodies that specifically detect HLA-DR, MerTK, MMP8, CD64, CD24, and CX3CR1.

Another preferred kit contains all the means to distinguish between a sepsis-2 or sepsis-3, that is namely antibodies that specifically detect HLA-DR, PCT, and IL-6. Another preferred kit contains or consists essentially in antibodies that specifically detect HLA-DR, hyaluronan, and creatinine. Another preferred kit contains or consists essentially in antibodies that specifically detect HLA-DR, PCT, IL-6, hyaluronan and creatinine.

Another preferred kit contains or consists essentially in antibodies that specifically detect HLA-DR, MerTK, MMP8, CD64, CD24, CX3CR1, PCT, IL-6, hyaluronan and creatinine, so as to detect a bacterial/viral infection, and diagnose subjects suffering from sepsis-2 or -3, as proposed above.

These kits can also contain all the means that are useful for performing appropriate analytical technologies such as cell membrane staining, immunoprecipitation, flow cytometry, western blot, ELISA, ELISPOT, antibodies microarrays, immunohistology, dot blot, protein microarray, or tissue microarrays coupled to immunohistochemistry.

### Methods for Treatment

In another aspects, the invention encompasses methods for treatment. In some embodiments, the method further comprises administering an antimicrobial or antiviral agent to a patient that has been diagnosed to suffer from a bacterial or viral infection respectively.

The treatment administered is determined based on the diagnosis defined by the use if the biomarkers described herein.

Specifically, the present invention encompasses the antibacterial agents such as those disclosed herein, for use for treating patients that display a decreased level of HLA-DR (human leukocyte antigen DR) expression on the monocytes, an increased level of MerTk (Myeloid-epithelial-reproductive tyrosine kinase) expression on the neutrophils, and an increased level of Metalloproteinase-8 (MMP8) in the plasma, as compared to reference levels.

Also, the present invention encompasses antiviral agents such as those disclosed herein, for use for treating patients that display a decreased level of CD64 expression on the neutrophils, a decreased level of CD24 expression on the neutrophils, and an increased level of CX3CR1 expression on the monocytes as compared to reference levels.

In other words, the present invention encompasses methods to select a therapy in view of the results of the methods of the invention (if the patient is diagnosed with the method of the invention to suffer from a bacterial infection, then the therapy will be an antibiotic agent known in the art; whereas if the patient is diagnosed with the method of the invention to suffer from a viral infection, then the therapy will be an antiviral agent known in the art).

An antibiotic agent can be selected from one or more of amikacin, gentamicin, kanamycin, neomycin, netilmicin, tobramycin, paromomycin, geldanamycin, herbimycin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefditoren, cefoperazone, cefotaxime, cefpodoxime, ceftazidime, ceftibuten, cenizoxime, ceftriaxone, cefepime, ceftaroline fosamil, ceftobiprole, teicoplanin, vancomycin, telavancin, clindamycin, lincomycin, daptomycin, azithromycin, clarithromycin, dirithromycin, erythromycin, roxithromycin, troleandomycin, telithromycin, spectinomycin, spiramycin, aztreonam, furazolidone, nitrofurantoin, amoxicillin, ampicillin, azlocillin, carbenicillin, cloxacillin, dicloxacillin, mezlocillin, methicillin, nafcillin, oxacillin, penicillin G, penicillin V, piperacillin, temocillin, ticarcillin, amoxicillin/clavulanate, ampicillin/sulbactam, piperacillin/tazobactam, ticarcillin/clavulanate, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine, sulfacetamide, sulfadiazine, silver sulfadiazine, sulfamethizole, sulfamethoxazole, sulfanilimide, sulfasalazine, sulfisoxazole, trimethoprim, trimethoprim-sulfamethoxazole, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, linezolid, metronidazole, mupirocin, platensimycin , quinupristin/dalfopristin , rifaximin , thiamphenicol, tigecycline, tinidazole, pharmaceutically acceptable salts thereof, derivatives thereof, and combinations thereof.

An antiviral agent can be selected from idoxuridine, trifluridine, pleconaril, rifampicin, fomivirsen, vidarabine, acyclovir, ganciclovir, valganciclovir, valacyclovir, cidofovir, famciclovir, ribavirin, amantadine, rimantadine, interferon, oseltamivir, palivizumab, rimantadine, zanamivir, peramivir, nucleoside-analog reverse transcriptase inhibitors (nrti) such as zidovudine, didanosine, zalcitabine, stavudine, lamivudine and abacavir, non-nucleoside reverse transcriptase inhibitors (nnrti) such as nevirapine, delavirdine and efavirenz, protease inhibitors such as saquinavir, ritonavir, indinavir, nelfinavir, amprenavir, and other known antiviral compounds and preparations.

### Definitions

The methods of the invention can be performed on any animal, in particular human beings. Therefore, as used herein, the term “subject” designates any animal (e.g., cats, dogs, horse, cattle, etc.), and also includes human beings. The term “patient” usually refers to human subjects, more particularly to human subjects that have been accepted in a hospital ED.

In the present methods, the biological sample is preferably a blood sample, a plasma sample. Since monocytes, neutrophils, calprotectins and cytokines are mostly found in the blood and plasma, it is particularly advantageous to use blood and/or plasma as a biological sample for the method of the invention. Indeed, such a blood and/or plasma sample may be obtained by a completely harmless, non-invasive blood and/or plasma collection from the subject. The blood and/or plasma sample used in the present methods is preferably depleted of most, if not all erythrocytes, by common red blood cell lysis procedures. The detection is performed on the remaining blood cells, which are white blood cells (e.g., neutrophils, monocytes, lymphocytes, basophiles, etc.) and platelets.

As used herein, the terms “blood sample” or “sample” refer to any blood sample which may contain monocytes and/or neutrophils, including, but not limiting to, whole blood (e.g., non-purified, raw blood) or blood plasma. More preferably, the biological sample is a peripheral blood sample. Indeed, such a blood sample may be obtained by a completely harmless and non-invasive blood collection from the subject.

Any volume used commonly by the person of skills in the art for hematological analyses will be convenient for the present methods. For example, the volume of the blood sample can be of 100 μL, 200 μL, 300 μL, 400 μL, 500 μL, 600 μL, 700 μL, 800 μL, 900 μL, or 1000 μL (1 mL).

As used herein, a “biomarker” or a “biological marker” is a measurable indicator of a biological state, condition, ailment, disease and form/stage thereof. A biomarker can be a substance whose detection indicates a particular disease state, for example, the presence of an antigen or cellular receptor (or a cell associated therewith) may indicate an infection. It can also be used to optimize a treatment/therapy, and to evaluate the likelihood of benefiting or the benefice from a specific therapy, and can serve a role in narrowing down diagnosis.

In the methods of the invention, expression of cell surface receptors and/or cell surface antigens (on any cells, including blood and/or plasma cells, such as monocytes, neutrophils) may be notably assessed (measured, or identified, or detected) using well known analytical technologies such as cell membrane staining using biotinylation or other equivalent techniques followed by immunoprecipitation with specific antibodies, flow cytometry, western blot, ELISA, ELISPOT, antibodies microarrays, immunoprecipitation, immunohistology, dot blot, protein microarray, or tissue microarrays coupled to immunohistochemistry. Other suitable techniques include FRET or BRET, single cell microscopic or histochemistery methods using single or multiple excitation wavelength and applying any of the adapted optical methods, such as electrochemical methods (voltametry and amperometry techniques), atomic force microscopy, and radio frequency methods, e.g. multipolar resonance spectroscopy, confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), cell ELISA, radioisotopic, magnetic resonance imaging, analysis by polyacrylamide gel electrophoresis (SDS-PAGE); HPLC-Mass Spectroscopy; Liquid Chromatography/Mass Spectrometry/Mass Spectrometry (LC-MS/MS)). For example, when flow cytometry is used, forward scatter and side scatter information help to identify the monocyte population among other blood cells. Preferably, the cells (including blood and/or plasma and/or bone marrow cells, such as monocytes, neutrophils, lymphocytes and leukocytes, as defined above) are identified, selected, sorted, quantified (and any combination thereof) by flow cytometry.

The terms “reference value”, or “reference quantity” or “reference level” as used herein, refers to the value (or quantity, or level) of a parameter or a biomarker indicating the state of a subject with respect to a specific disease (or ailment, or condition). The suitable reference level of a parameter or a biomarker can be quantified, or determined, or measured by detecting the parameter/biomarker in several suitable reference subjects. Such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference level can be based on a large number of samples, such as from population of subjects of the chronological age matched group, or based on a pool of samples including or excluding the sample to be tested. Depending on the context, the reference level corresponds to the value of a parameter (or a biomarker) quantified, or determined, or measured, on a sample from a healthy reference subject; or to the average of the values (mean value) of a parameter (or a biomarker) quantified, or determined, or measured, on different samples of the same healthy reference subject (values quantified/determined/measured on samples taken at separate time intervals from the same healthy reference subject); or to the average of the values (mean value) of a parameter/biomarker determined/measured on the same sample from a healthy reference subject but at separate time intervals; or to the average of the values (or mean value) of a parameter/biomarker quantified/determined/measured on samples from several healthy reference subjects (at least two healthy reference subjects).

Alternatively, the reference level can correspond to the value of a parameter (or a biomarker) quantified, or determined, or measured, on a sample from a reference subject that does not suffer from viral infection nor from bacterial infection; or to the average of the values (mean value) of a parameter (or a biomarker) quantified, or determined, or measured, on different samples of such a reference subject; or to the average of the values (mean value) of a parameter/biomarker determined/measured on the same sample from such a reference subject but at separate time intervals; or to the average of the values (or mean value) of a parameter/biomarker quantified/determined/measured on samples from several reference subjects (at least two reference subjects) that do not suffer from viral infection nor from bacterial infection.

In the present invention, the biomarkers can be particular proteins that have to be detected on the surface of particular blood cells. In this case, the measuring of “increased” or “decreased” expression levels of said surficial biomarkers (HLA-DR, MerTK, CD64, CD24, CXCR1) is preferably performed by measuring the “increased” or the “decreased” level of expression of these surficial proteins, for example by flow cytometry.

Alternatively, it is possible to measure “increased” or “decreased” expression levels of said surficial biomarkers (HLA-DR, MerTK, CD64, CD24, CXCR1) by measuring the “increased” or the “decreased” amount of said particular cells expressing high levels of these surficial proteins, for example by flow cytometry. The exact amount of said surficial proteins is not important, what matters is to compare the number of cells expressing a sufficient amount of said proteins to be detected (therefore expressing “high” amount of said proteins) with the number of the same cells expressing high amount of said proteins in the reference sample. In other terms, the actual quantitative “level of expression” of the surficial biomarkers is not necessarily measured, it is preferred to measure the number of cells expressing “high” or “low” amount of the biomarkers. Said number of cells will then be compared to the number of cells expressing “high” or “low” amount of the biomarkers in the reference sample.

The term “monocytes” herein refers to a type of leukocytes (representing 2 to 10% of circulating leukocytes, 0.1 to 1×10⁹/L in human peripheral blood) produced by the bone marrow from hematopoietic stem cells. They circulate in the blood, typically between one and 7 days, and most of them migrate into tissues where they differentiate, generating so-called “monocyte-derived cells” with a macrophage phenotype. Monocytes belong to the family of the peripheral mononuclear cell of the blood (PBMCs). PBMCs are a critical component in the immune system to fight infection and adapt to intruders. These cells can be extracted from whole blood using Ficoll, a hydrophilic polysaccharide that separates layers of blood, which will separate the blood into a top layer of plasma, followed by a layer of PBMCs and a bottom fraction of polymorphonuclear cells (such as neutrophils and eosinophils) and erythrocytes. Monocytes are fairly variable in size and appearance, but they show common expression of a number of markers, including cell surface antigens (or receptors). Three subsets of monocytes can be identified in human blood, based on the expression of the CD14 and CD16 markers: a) the “classical” monocyte or MO1 (as used herein “classical monocyte”) is characterized by high level expression of the CD14 cell surface receptor and no expression of CD16 cell surface receptor (CD14⁺/CD16⁻ monocyte or CD14^{high}/CD16^{low}), b) the “non-classical” monocyte or MO3 (as used herein “non-classical monocytes”) shows low level or no expression of CD14 with additional co-expression of the CD16 receptor (CD14⁻/CD16⁺ monocyte or CD14^{low}/CD16^{high}), and c) the “intermediate” monocyte or MO2 (as used herein “intermediate monocyte”) with high level expression of CD14 and the same level of CD16 expression as the MO3 monocytes (CD14⁺/CD16⁺ monocytes or CD14^{high}/CD16^{high}) ([13], [16], [17]).

Monocytes are easily identified by specific antigens (including cell surface antigens, e.g. CD14 or CD16) combined with morphometric characteristics (e.g. size, shape, granulometry, etc.).

Thus, most of the monocytes, like classical monocytes, express the “cluster of differentiation CD14” or “CD14” or “CD14 molecule/antigen”. The amino acid sequence of reference for the human CD14 is the NCBI sequence referenced under NP_000582.1. Numerous antibodies against human CD14 are commercially available. CD14 is expressed at the surface of the monocytic cells and, at 10 times lesser extent, of the neutrophils.

The “cluster of differentiation CD16” or “CD16” or “CD16 molecule/antigen” is the low affinity receptor for the Fc part of IgG (therefore also known as FcγRIII), is a glycoprotein expressed in monocytes, and also in NK cells and neutrophils. Two isoforms (A and B) exist. In human, the isoform A has the NCBI reference sequence NP_000560.5 and the isoform B has the NCBI reference sequence NP_001231682.1. Several monoclonal antibodies have been produced against the isoforms A and B of CD16/FcγRIII and the corresponding epitopes have been localized on these proteins (see e.g., [21]; [29]; [40]). Antibodies against CD16 are available commercially.

Monocytes, including classical monocytes, can also express the HLA-DR cell surface receptor. These monocytes are referred to as HLA-DR⁺ monocytes. HLA-DR is an MHC (major histocompability complex) class II cell surface receptor encoded by the human leukocyte antigen complex on chromosome 6 region 6p21.31. The complex of HLA-DR (Human Leukocyte Antigen—DR isotype) and peptide, generally between 9 and 30 amino acids in length, constitutes a ligand for the T-cell receptor (TCR). The primary function of HLA-DR is to present peptide antigens, potentially foreign in origin, to the immune system for the purpose of eliciting or suppressing T-(helper)-cell responses that eventually lead to the production of antibodies against the same peptide antigen. Antigen presenting cells (macrophages, B-cells and dendritic cells) are the cells in which DR are typically found. HLA-DR is an a6 heterodimer, cell surface receptor, each subunit of which contains two extracellular domains, a membrane-spanning domain and a cytoplasmic tail. The reference amino acid sequence for human HLA-DR alpha and beta chains can be represented by the NCBI accessions AAA36275.1 or AAA59785.1 or AAA36302.1 (alpha chain) and AAA58651.1 or AAA59816.1 (beta chain).

“Neutrophils”, also known as “neutrocytes” or “heterophils”, are the most abundant type of granulocytes and the most abundant (60% to 70%) type of white blood cells in most mammals. They form an essential part of the innate immune system, with their functions varying in different animals. These specialized innate cells require constant replenishment from proliferative bone marrow precursors as a result of their short half-life. Neutrophils undergo a process called chemotaxis, which allows them to migrate toward sites of infection or inflammation. Neutrophils have a variety of specific receptors, including ones for complement, cytokines like interleukins and IFN-γ, chemokines, lectins, and other proteins. They also express receptors to detect and adhere to endothelium and Fc receptors for opsonin. The antigens expressed by neutrophils include, but are not limited to, CD10, CD11b, CD15, CD16, CD35, CD64, CD66a, CD66b, CD101, CD111, CD177, etc. They can be distinguished from other white blood cells by the expression of CD177 and/or CD15+CD66b+.

As used herein, “contaminant cells” or “contaminant white blood cells” refer to the cells or the white blood cells which are present in the biological sample (advantageously a blood sample) of the subject and which are not monocytes and/or neutrophils (depending on the targeted biomarkers). Such contaminant cells include eosinophils, basophils, and lymphocytes, e.g., T cells, NK cells, B cells, but also precursors of these cell types.

In a particular embodiment, it is advantageous to detect a substantially pure monocyte population, that is, a population of monocytes that is devoid of contaminant cells. The remaining white blood cells can be identified and counter-selected on the basis of the expression of specific markers. Using anti-CD15, anti-CD16, anti-CD56, anti-CD2 or anti-CD24 antibodies enables to detect and therefore exclude, if needed, the cells expressing CD2, CD56 and CD24 proteins, notably the CD2+ T lymphocytes, the CD2+ NK cells, the CD56+ NK cells, the CD24+ immature granulocytes as well as the CD15+ or CD16++ granulocytes.

The existence of markers (including cell surface receptors/antigens) which are specific for each of the contaminant cell types enables the identification of these cells in the blood sample of the subject.

Identified contaminant cells can then be removed from the sample (i.e., physically) or from the analysis (i.e., by retaining only the data pertaining to the monocyte population for the analysis), so that the study then only focuses on the monocyte population. In this respect, although any of the above-mentioned analytical techniques can be used to identify the said contaminant white blood cells, flow cytometry is particularly adapted for this task, since it enables the skilled person to eliminate the contaminants and analyse the monocyte population with minimal effort. In particular, flow cytometry (especially exclusion gating by flow cytometry) can be performed with antibodies specific for well-known antigens expressed by granulocytes (CD24, CD15, CD16), T lymphocytes (CD2, CD3), B lymphocytes (CD24, CD19), and/or NK cells (CD2 and/or CD56), so that to discard these contaminant cells and retain only monocytes and/or neutrophils (depending on the targeted biomarkers). Using anti-HLA-DR, anti-CD10, anti-CD101, anti-CD15, anti-CD56, anti-CD2 or anti-CD24 antibodies therefore enables to detect and thus exclude the cells expressing CD2, CD10, CD101, CD15, CD56 and CD24 proteins, notably the CD2+ T lymphocytes, the CD2+ NK cells, the CD56+ NK cells, the CD24+ immature granulocytes as well as the CD15+ granulocytes, (if needed) the HLA-DR+ monocytes and/or (in needed) the CD10+CD101+ cells.

As used herein, “selecting a therapy” or “selecting a treatment” or “selecting a drug” refers to the process of selecting (choosing, or deciding for, or opting for) the most appropriate therapy for a subject, in view of the symptoms (or signs) detected (observed and/or measured) in the subject, and/or in view of the subject himself, and the general knowledge in the medical field (preferably the medical field closest to the disease). Selecting a therapy include selecting the most appropriate therapy and may include also selecting the most appropriate administration mode and/or the most appropriate posology.

### BRIEF DESCRIPTION OF THE DRAWINGS

FIG. 1 shows the list of biomarkers simultaneously investigated in this study. Abbreviation: CRP: C reactive protein, PCT: procalcitonin; MMP8: metalloproteinase-8; suPAR: soluble utokinase-type plasminogen activator receptor; TNFa: tumor necrosis factor-a; IL-: interleukin; IFN: interferon; CCL: Chemokine CCL; CXCL: Chemokine CXCL; PDGFb: platelet growth factor-b; ANG-2: angiopoietin-2; HNL: human neutrophil lipocalin.

FIG. 2A-D shows the individual values of the most relevant biomarkers according to the group of interest: (A) bacterial infection, no bacterial infection, (B) viral infection, no infection, (C) Sepsis-2, no sepsis, (D) Sepsis-3, no sepsis. Box plots indicate the distribution of values and dots the individual values.

FIG. 3A-D shows the Receiver Operating Characteristics (ROC) curve and Area Under The Curve (AUC) values for the combination of three biomarkers for the diagnosis of bacterial infection (A), viral infection (B), sepsis-2 (C) and sepsis-3 (D).

FIG. 4 discloses an exemplary flow cytometry gating strategy.

FIG. 5A-D shows the respective strength of each biomarker in the combinatory approach in identifying the groups of interest. (A): bacterial infection (B): viral infection (C): sepsis-2 (D): sepsis-3. Boxplots represent the distribution of the 15 selected markers in each group of interest and dots the individual values.

FIG. 6 discloses alternative combinations for bacterial infection group diagnosis.

FIG. 7 discloses alternative combinations for viral infection group diagnosis.

FIG. 8 discloses alternative combinations for sepsis-2 group diagnosis.

FIG. 9 discloses alternative combinations for sepsis-3 group diagnosis.

### EXAMPLES

The experimental details explained below are more completely disclosed in Velly L. et al, 2021 ([52]), which is incorporated herein by reference.

### Patients and Methods

### Setting and Inclusion Criteria

The BIPS study was conducted in the ED of an adult academic urban tertiary 1700-bed hospital, totalizing 69,000 visits a year. In order to gather the main situations where ED's physicians suspect infection or sepsis (and may benefit from the added value of biomarkers) the DELPHI method was used to define the criteria of inclusion [5] [6]. Briefly, the Delphi survey method is a procedure for building an expert consensus first developed for the field of forecasting by the Research ANd Development (RAND) Corporation in the 1950s. Since that time, it has been used widely for consensus building in many different areas of expertise including medical practice. The method involves the recruitment of a panel of experts in the field of interest and an iterative process whereby the panellists are asked to validate (agree or disagree) with a series of statements. Between each round, the statements are adapted so that panellists converge over consecutive rounds to reach a consensus. In this study, the panellists were national academic emergency physicians to whom a list of clinical and biological signs was submitted. They were asked to agree or disagree on each as a criterion to suspect sepsis. Two rounds were necessary to reach a consensus of criteria of inclusion (Table 1).

TABLE 1
clinical and biological inclusion criteria retained, according to DELPHI
method for the suspicion of infection or sepsis in emergency setting.
To be eligible for inclusion, patients should meet at least one criterion.

Temperature >38° C.
Chills
Marbling
Systolic blood pressure <90 mmHg or mean arterial pressure <70 mmHg
Cough
Purulent sputum
Unilateral thoracic altered sound on auscultation
Abdominal defense or contracture
Monoarthritis
Unilateral red swelling leg
Unilateral facial edema
Urine test strip positive for leukocytes and/or nitrite
White blood cells count <4000 or >12,000/mm³
Lactate >2 mmol/L

Patients 18-year-old and older were recruited consecutively and were given and had to sign informed consent before inclusion. The BIPS study was approved by the Ethic Committee and has been registered in ClinicalTrials (NCT02707718).

### Exclusion Criteria

We excluded patients <18-year-old, patients with no social insurance, homeless people without possible follow-up, pregnant women, refusal to participate, prisoners, dementia and/or cognitive impairment precluding informed consent signed. Similarly, patients with documented appendicitis or malaria were secondarily excluded because of specific pathophysiology and dedicated diagnostic procedures.

### Clinical and Biological Data, Follow-Up and Adjudication

The data collected were those registered during ED visits and comprised: age, sex, vital signs at nurse triage, ongoing anti-infective treatment, immune status (immunodeficiency defined by at least one among: cancer on chemotherapy immunosuppressive treatment, >15 mg/day prednisolone, human immunodeficiency virus infection, solid organ transplant), usual biological parameters analyzed, final diagnosis, orientation decision (outpatient, medical wards or intensive care unit hospitalization) and anti-infective treatment administered. The patient's care was totally at the discretion of the treating emergency physician. Microbiological investigations during hospital stay and ward's medical files were also recorded. A follow-up phone call was organized at day-30 to assess vital status and confirm the diagnosis for the patients discharged home after ED visit. All anonymized data (except the results of the biomarkers tested) were gathered into an excel sheet and for each patient included were submitted to an adjudication committee of 3 physicians: one emergency physician, one intensivist and one infectious disease specialist. Each adjudicator independently had to classify the patients into the pre-specified sepsis-2 and sepsis-3 criteria and- in case of infection -into bacterial, parasitic or viral infection [1,7]. Briefly, study subjects were categorized based on the Sepsis-2 consensus criteria as SIRS (≥2 SIRS criteria), sepsis (infection plus SIRS) (including sepsis [no organ failures], severe sepsis [sepsis with one or more organ failures], and septic shock [sepsis with refractory hypotension]), and infection but no sepsis (i.e., zero or one SIRS criterion), and based on the Sepsis-3 criteria such as no infection, infection, and sepsis (based on SOFA score criteria). The diagnosis of infection was determined based on the retrospective chart review of tests performed and clinical data available. Test results were extracted from the records 7-10 days later, including cultures, molecular tests (e.g., polymerase chain reaction and antigens), relevant imaging, and tissue pathology. In case of no consensus could be reached, a fourth independent physician opinion was requested to arbitrate.

### Sampling

Blood collection for biomarkers measurement (3×4 ml EDTA tubes) was performed by the ED nurses at the first venous blood sampling during the initial care of the patient, and before any significant therapeutic intervention. The patients were screened and enrolled on week days only on morning, to allow sufficient time for sample processing on the same day (cytometric analyzes without fixation).

Immediately after collecting the venous blood, two tubes were kept at +4° C. until transportation on ice every day to Institut Pasteur, where expression of cell surface markers was assessed directly on whole blood by flow cytometry, and plasma isolated by centrifugation was stored at −80° C. until plasma biomarkers measurement. The delays between blood draw and flow cytometry analysis, plasma collection and freezing were 4 to 8 hours, 2 to 6 hours and 4 to 8 h, respectively. During these delays, the samples were kept at +4° C. since blood collection, excepted for one tube that was kept at room temperature in the ED during one to two hours for human neutrophil lipocalin (HNL) dosage (as recommended by the manufacturer to improve and standardize the release of HNL by neutrophils at room temperature) then plasma was isolated by centrifugation and stored at +4° C. before transportation on ice to the Institut Pasteur every day.

### Biomarker's Selection

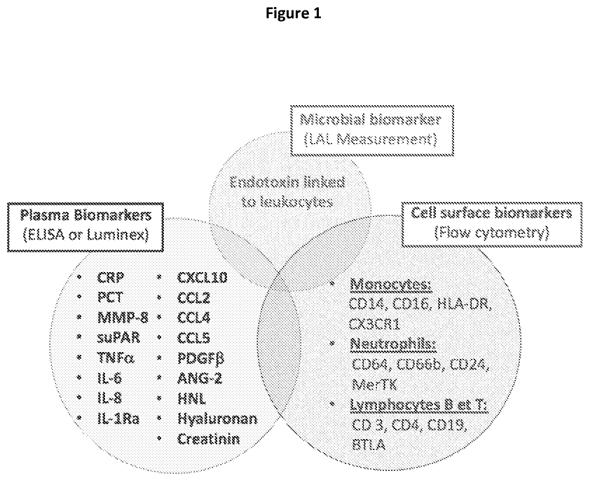

In order not to bias the selection of the biomarkers tested, it was decided to incorporate into the panel all the biomarkers that had been already reported almost twice in the literature as potentially promising for sepsis and/or infection diagnosis [8-25]. Eighteen different biomarkers measured in plasma, and twelve biomarkers measured on monocytes, neutrophils, B and T-lymphocytes were included; as well as a bacterial biomarker (endotoxin linked to leukocytes) (FIG. 1).

### Methods of Measuring Biomarkers

For plasma biomarkers, two samples were centrifuged in the ED to obtain plasma, and stored at 4° C. until freezing. Plasma was transferred into 0.5-1 mL aliquots tubes (Eppendorf Biopur) and stored at −80° until assayed.

Plasma biomarkers were measured using an enzyme linked immunosorbent assay or fluorescence immunoassay: ELISA HNL/NGAL Diagnostics Development (Uppsala, Sweden), ELISA Angiopoietin2 Abcam (Cambridge, United Kingdom), ELISA Human MMP-8 R&D Systems (Minneapolis, USA), ELISA suParnotics AUTO Flex ELISA kit ViroGates (Birkerod, Denmark), Human Cytokine Assays-Bio-Plex Pro Assays BioRad (Hercules, USA), with an immunoassay analyzer. Interferon-alpha measurement was performed as described previously [26]. All measurements were performed according to the manufacturer's instructions.

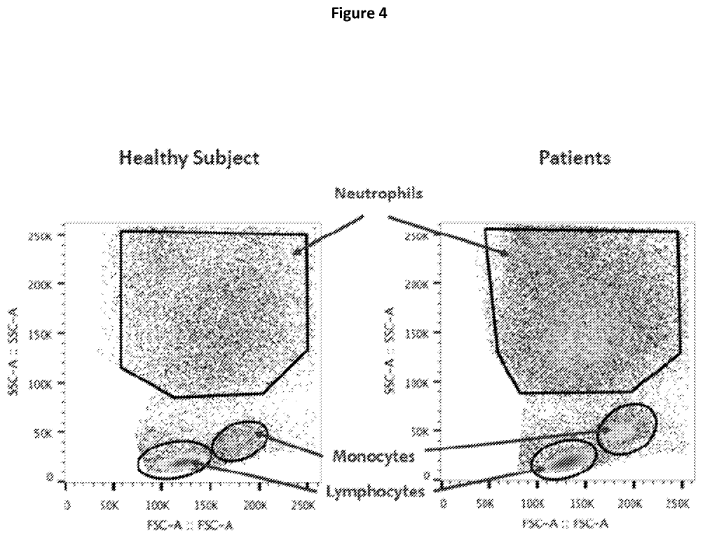

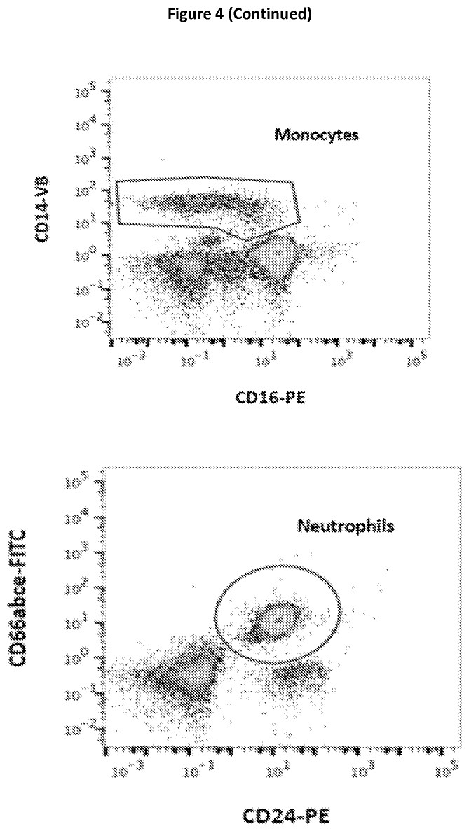

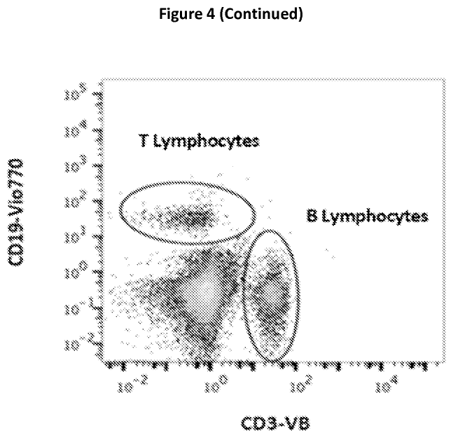

Expression of cell surface markers was assessed directly on whole blood by flow cytometry (MACSquant Miltenyi Biotec). The gating strategy is represented in FIG. 4. Anti-CD14-VB, anti-CD16-PE, anti-HLADR-PE/Vio770, anti-CX3CR1-AlexaFluor674; anti-CD64-VB, anti-CD66abce-FITC, anti-CD24-PE, anti-MerTK-AlexaFluor674, anti-CD3-VB, anti-CD4-FITC, anti-CD19-PE/Vio770, anti-BTLA(CD272)-APC were obtained from Miltenyi Biotec (CD14, CD16, HLADR, CD64, CD66abce, CD24, CD3, CD4, CD19: Bergisch Gladbach, Deutschland), BioLegend (CX3CR1, MERTK, San Diego CA, USA), and BD Biosciences (BTLA, San Jose, CA, USA). Whole blood (100 μL) was processed by FCR blocking (Miltenyi-10 min of incubation in the dark) and was stained with antibody (20 min of incubation in the dark); subsequently, 3 mL of lysis buffer (BioLegend) was added samples to lyse erythrocytes. After a 10 min incubation at +4° C. and centrifugation, the supernatant was treated by Viability Dye-eFluor780 (Invitrogen/eBioscience-ThermoFisher, CarlsBad, CA, USA) and after a new step of incubation at +4° C. and centrifugation the supernatant was removed and 300 μL of MACS buffer was added to cell. The expression of surface markers was immediately measured by flow cytometry. All the mAbs were used according to manufacturer's recommendation. Data analysis was performed using FlowJo software. Settings of the flow cytometer were maintained constant during the whole study. Values were expressed as mean fluorescence intensity (MFI) or percentage of expression.

For the endotoxin assay, cell isolation was performed. Briefly, a buffy coat was isolated by whole blood sedimentation on a Glucose-Dextran (separating leukocytes from the majority of red blood cells). Then leukocytes (neutrophils and mononuclear cells) were isolated by centrifugation on a Ficoll density gradient (MSL-Eurobio, France). Peripheral blood mononuclear cells were obtained in the first ring. Neutrophils were obtained in the second ring after a step of erythrocyte lysis. Freshly isolated neutrophils and mononuclear cells were resuspended in physiological serum, and five successive freezing and thawing cycles are performed at −20° C. After centrifugation at 10 000 g, the supernatants are transferred in tube (aliquot of 250 μl/tube-Eppendorf-Biopur). Test QCL1000-Limulus Amebocyte Lysate Biowhittaker-Lonza (Basel-Switzerland) was applied for the dosage of endotoxin binding on neutrophils and mononuclear cells. The LAL assay was used according to manufacturer's recommendation.

### Statistical Methods

Clinical and biological data are described as frequencies and percentages for categorical variables and as means and standard deviations or medians and interquartile ranges for continuous variables, as appropriate. To identify the biomarkers which may discriminate pre-defined groups of patients (bacterial infection, viral infection, sepsis) a gradient boosting tree approach (xgbTree function from the caret R package v6.0.3.81, http://cran.r-project.org) was applied. Gradient Boosting Tree is a machine learning approach which maximizes the accuracy of the prediction by progressively training more complex models. All the models are combined to obtain the predictions. This process helps to reduce bias and variance. The final estimation depends on a set of hyperparameters which are tuned according to the accuracy (default option in caret package) [27]. The 15 most discriminating markers (including clinical and routine biological variables) were selected using a Mann Whitney test as a preliminary filtering step. The size of the training and testing sets was 262 and 29, respectively. All the combinations of the 15 most discriminating markers (2¹⁵−1 combinations) were explored to determine the best one according to the receiver operating characteristic area under the curve (AUC) criterion. To avoid over-fitting, a 10-fold cross-validation process was performed. All the AUCs were calculated on the test samples. It was aimed at identifying a biomarker or combination of biomarkers with an AUC >0.9 for the main criteria of judgment. Estimating that one third of included patients would fullfill this criteria, 280 patients had to be recruited with an alpha risk of 5% and to obtain an AUC's 95% confidence interval of 0.1.

### Results

From March 2016 to July 2017, 308 consecutive patients suspect of infection or sepsis were included: 6 patients were excluded for missing blood samples, 3 for acute appendicitis and 8 for malaria. Further analysis was performed on the 291 remaining patients. Median age was 60 years (interquartile range IQR, 32), and 53.6% were women. The baseline characteristics of the cohort are represented in Table 2.

TABLE 2
study participants baseline characteristics and outcome,
according to the different endpoints. N: number. IQR: interquartile range.

| | | Bacterial | Viral | No | | | Infection |
|---|---|---|---|---|---|---|---|
| | All patients | Infection | infection | infection | Sepsis-2 | Sepsis-3 | no sepsis |
| Characteristics | (n = 291) | (n = 148) | (n = 47) | (n = 96) | (n = 70) | (n = 16) | (n = 78) |
| Sex (%) | | | | | | | |
| Men | 46.4 | 39.9 | 57.4 | 51 | 38.6 | 25 | 47.2 |
| Women | 53.6 | 60.1 | 42.6 | 49 | 61.4 | 75 | 52.8 |
| Age, y | 60 | 60 | 58 | 58 | 62 | 65 | 59 |
| Median (IQR) | (42-73) | (40-73) | (42-72) | (50-75) | (48-73) | (57-72) | (39-73) |
| Systolic blood | 130 | 123 | 140 | 140 | 123 | 101 | 131 |
| pressure, | (114-147) | (108-143) | (126-150) | (121-151) | (105-143) | (88-139) | (114-144) |
| median (IQR), | | | | | | | |
| mm Hg | | | | | | | |
| Heart rate, | 95 | 98 | 98.5 | 99 | 104 | 104 | 94 |
| median (IQR), | (82-108) | (87-110) | (85-106) | (76-104) | (94-118) | (82-115) | (83-104) |
| mmm Hg | | | | | | | |
| Temperature, | 37.1 | 37.4 | 37.8 | 37.8 | 37.9 | 37.3 | 37.3 |
| median (IQR), | (36.5-38.0) | (36.8-38.2) | (36.6-38.2) | (36.4-37.2) | (36.9-38.6) | (36.9-38.2) | (36.6-38.0) |
| ° C. | | | | | | | |
| Immuno- | 52 | 27 | 9 | 16 | 17 | 5 | 12 |
| compromised | (17.9) | (18.2) | (19.1) | (16.7) | (24.3) | (31.2) | (15.2) |
| No. (%) | | | | | | | |
| White blood | 10.37 | 13.0 | 7.9 | 8.88 | 14.08 | 15.97 | 10.6 |
| cell count, | (7.75-14.5) | (9.98-17.11) | (6.65-11.89) | (7.03-11.73) | (10.43-18.2) | (13.0-19.15) | (7.8-13.8) |
| Giga/L | | | | | | | |
| Polymorphonu | 7.99 | 10.39 | 5.69 | 6.53 | 11.27 | 13.83 | 7.88 |
| clear Giga/L | (5.31-11.63) | (7.20-14.46) | (4.38-9.49) | (4.50-8.21) | (8.84-15.72) | (11.47-17.94) | (5.24-11.58) |
| Lymphocytes | 1.14 | 1.08 | 1.03 | 1.36 | 0.92 | 0.61 | 1.18 |
| Giga/L | (0.76-1.73) | (0.69-1.64) | (0.62-1.44) | (0.97-1.97) | (0.60-1.45) | (0.51-0.78) | (0.74-1.72) |
| Creatinin | 74 | 78 | 68 | 70 | 82 | 151 | 69 |
| microgr/L | (59-91) | (63-97) | (57-88) | (56-85) | (66-126) | (114-201) | (56-87) |
| Patient’s | 120 (41.2) | 39 (26.4) | 22 (46.8) | 61.5 | 12 (17.1) | 0 (0.0) | 30 (38.5) |
| course after | 22 (7.6) | 10 (6.8) | 2 (4.3) | 10.4 | 8 (11.4) | 6 (37.5) | 3 (3.8) |
| ED visit No. | 149 (51.2) | 99 (66.9) | 23 (48.9) | 28.1 | 50 (71.4) | 10 (62.5) | 45 (57.7) |
| (%) | | | | | | | |
| Non admitted | | | | | | | |
| ICU | | | | | | | |
| Medical or | | | | | | | |
| surgical wards | | | | | | | |
| Deceased at | 15 (5.2) | 11 (7.4) | 1 (2.1) | 3 (3.1) | 8 (11.4) | 4 (25) | 3 (3.8) |
| day-30 No. (%) | | | | | | | |
| Hemoculture | | | | | | | |
| No. (%) | | | | | | | |
| Positive | 26 (8.9) | 19 (12.8) | 0 | 0 | 14 (20) | 5 (31.2) | 37 (4.8) |
| Gram | 14 (53.8) | 9 (47.4) | — | | 6 (43) | 2 (40) | 25 (66.7) |
| + | 11 (42.3) | 9 (47.4) | — | | 7 (50) | 2 (40) | 12 (33.3) |
| Gram | 1 (3.8) | 1 (5.2) | — | | 1 (7) | 1 (20) | 0 |
| − | 130 (44.7) | 81 (54.7) | 29 | 27 | 42 (60) | 11 (68.8) | 42 (53.6) |
| Yeast | 135 (46.4) | 48 (32.4) | (61.7) | (28.1) | 14 (20) | | 32 (41.6) |
| Negative | | | 18 | 69 | | 0 | |
| Not performed | | | (38.3) | (71.9) | | | |
| Main sites of | | | | | | | |
| infection No. | | | | | | | |
| (%) | 84 (28.9) | 49 (33.1) | 35 | | 19 | 3 (18.7) | 30 (38.5) |
| Respiratory | 44 (15.1) | 44 (29.7) | (74.5) | | (27.1) | 6 (37.5) | 16 (20.5) |
| Urinary | 35 (12.0) | 28 (18.9) | 0 | | 22 | 4 (25.0) | 13 (16.6) |
| Pelviabdominal | 22 (7.6) | 21 (14.2) | 7 (14.9) | | (31.4) | 3 (18.7) | 3 (3.8) |
| | 6 (2.0) | 3 (2.0) | 1 (2.1) | | 15 | 0 | 0 |
| Cutaneous | 1 (0.3) | 0 | 3 (6.4) | | (21.4) | 0 | 0 |
| Head and | 3 (1.0) | 3 (2.0) | 1 (2.1) | | 8 (11.4) | 0 | 0 |
| neck | | | 0 | | 3 (4.3) | | |
| Neuromeningeal | | | | | 0 | | |
| Endocarditis | | | | | 3 (4.3) | | |

Bacterial infection was adjudicated for 148/291 (51%) patients, and viral infection for 47/143 (33%) patients with no bacterial infection. 70/291 (24%) patients were adjudicated as sepsis-2 and 16 (5%) as sepsis-3. The inter-rater reliability for the different endpoints (bacterial infection, viral infection, sepsis) was good (Kappa: 0.69) and a 4^{th} adjudicator (arbitrator) was requested in 3.2% of cases. Thirty-day mortality rate was 5.2%.

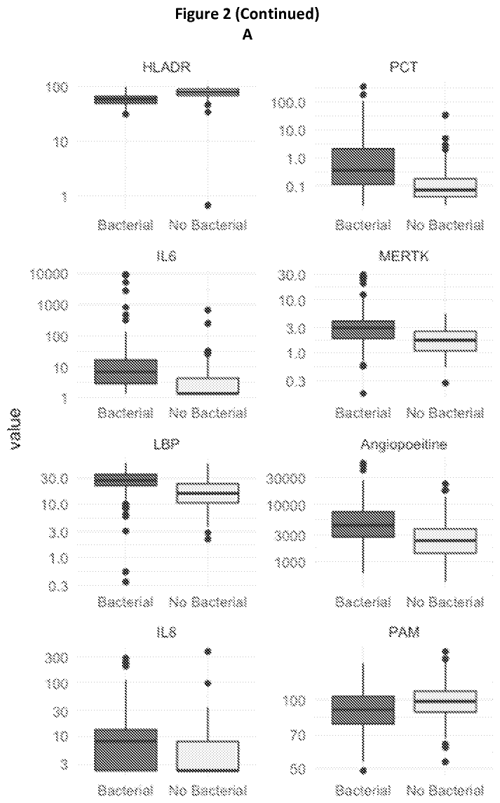

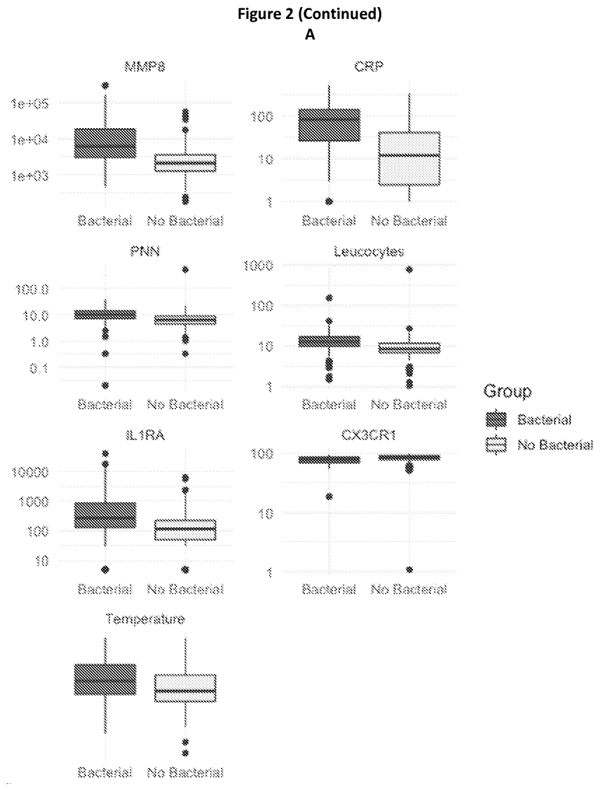

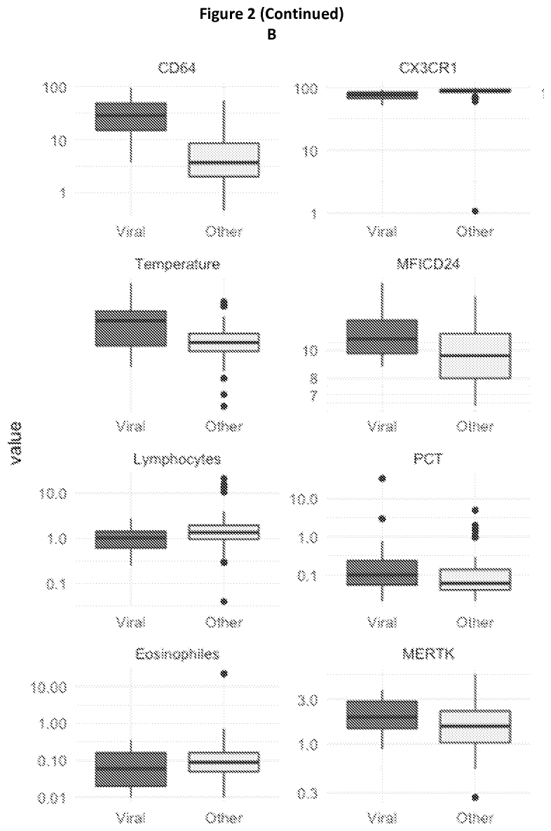

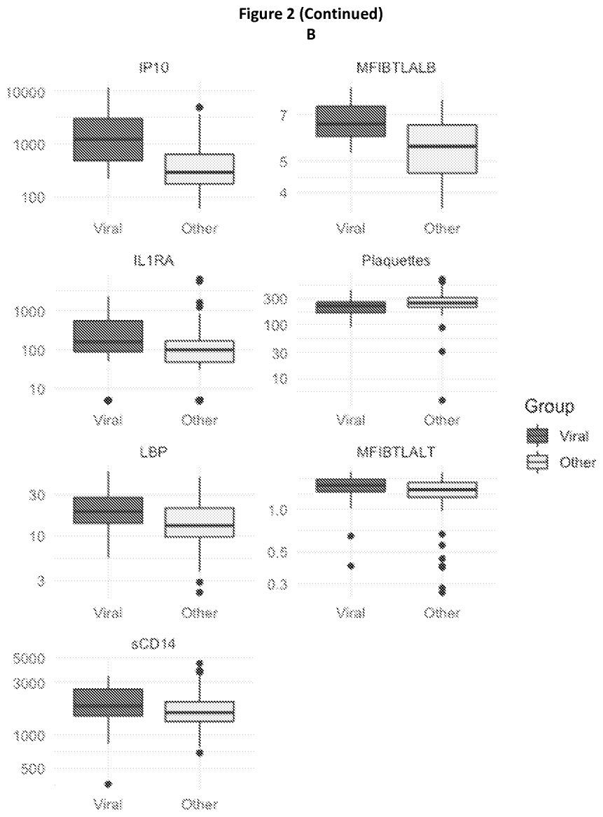

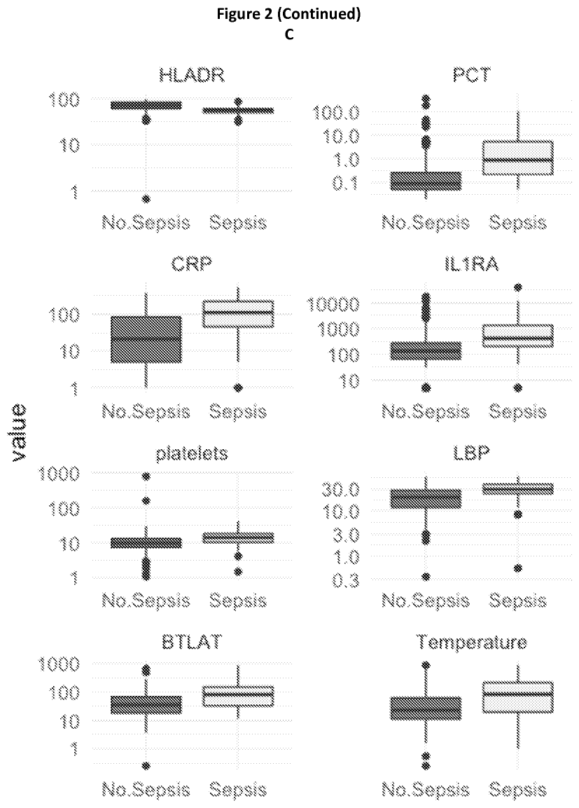

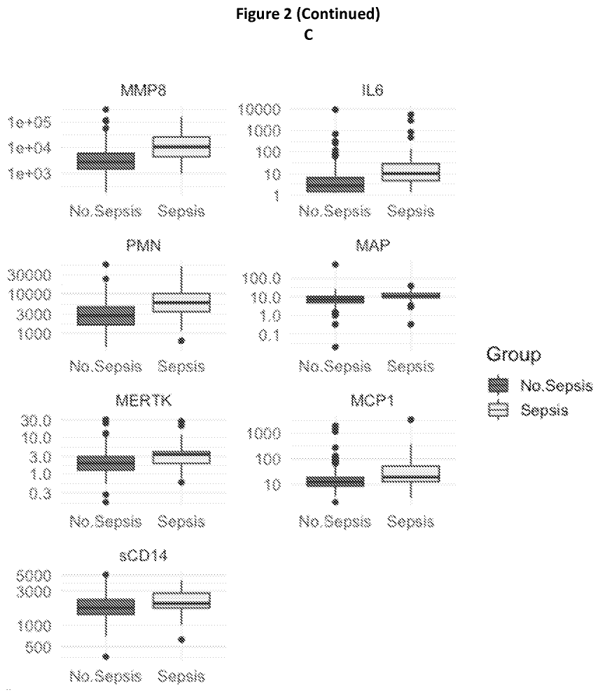

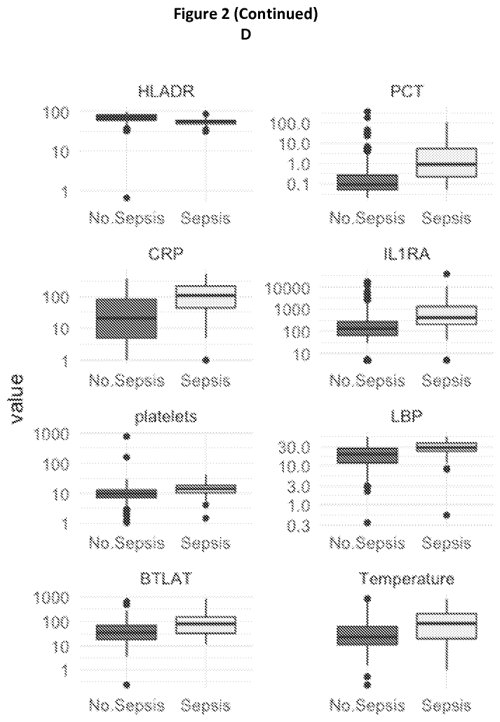

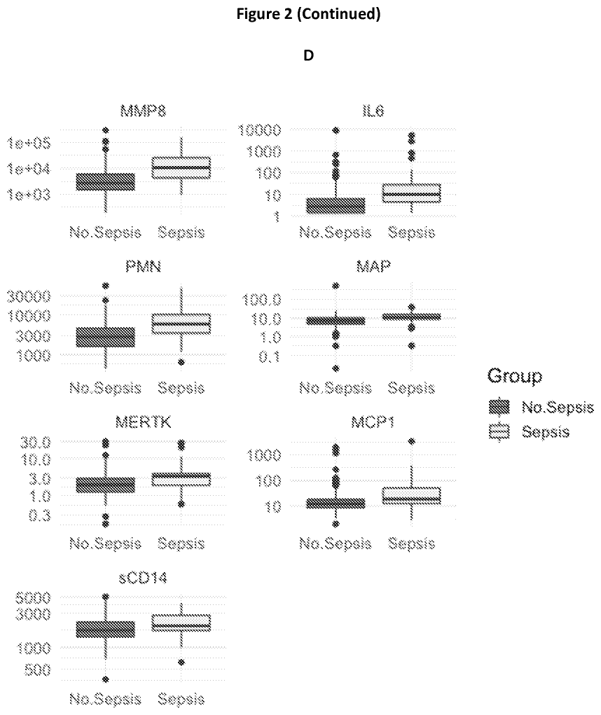

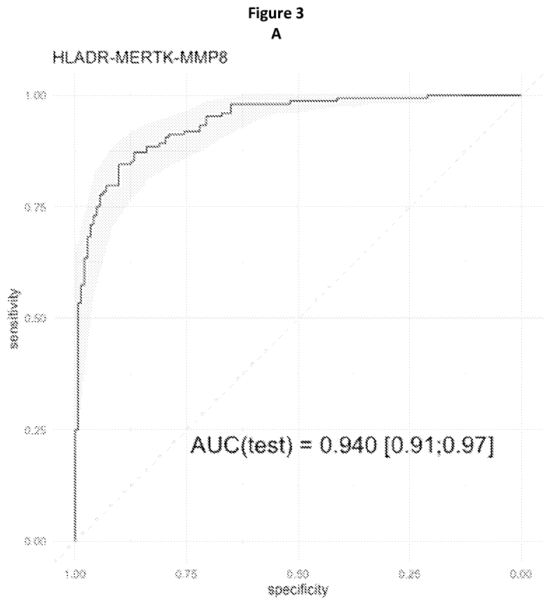

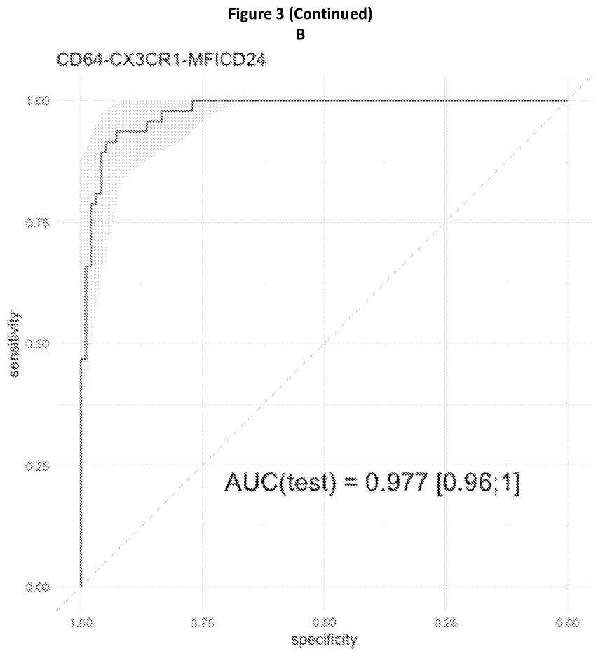

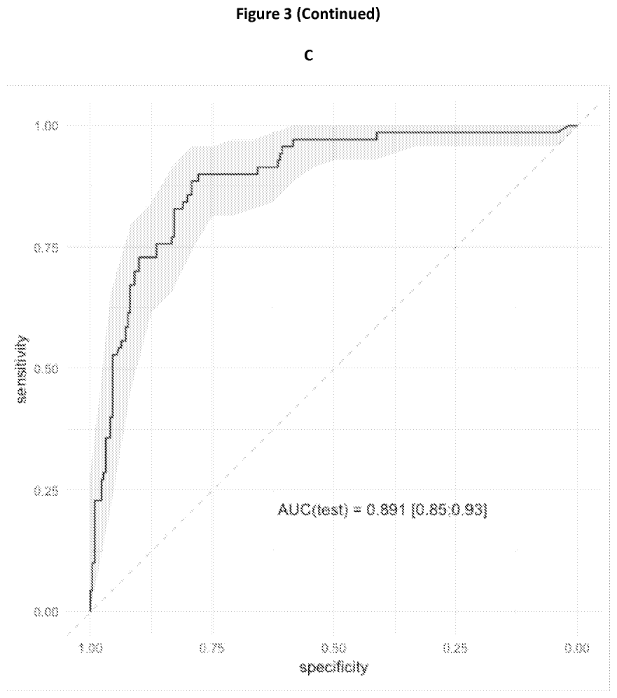

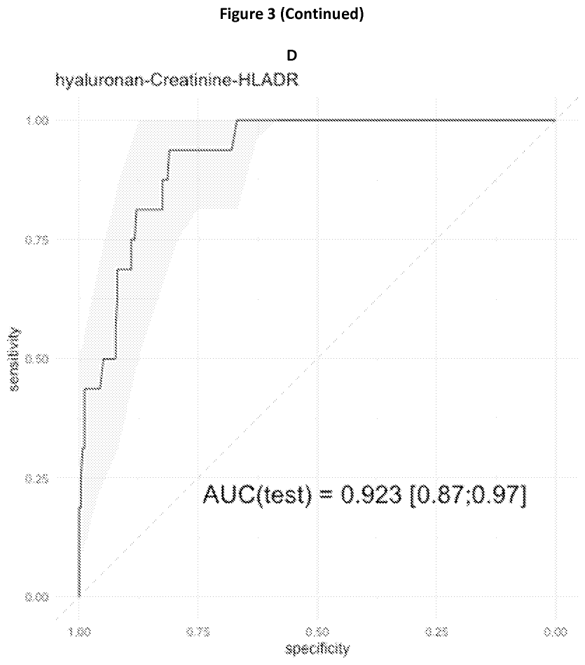

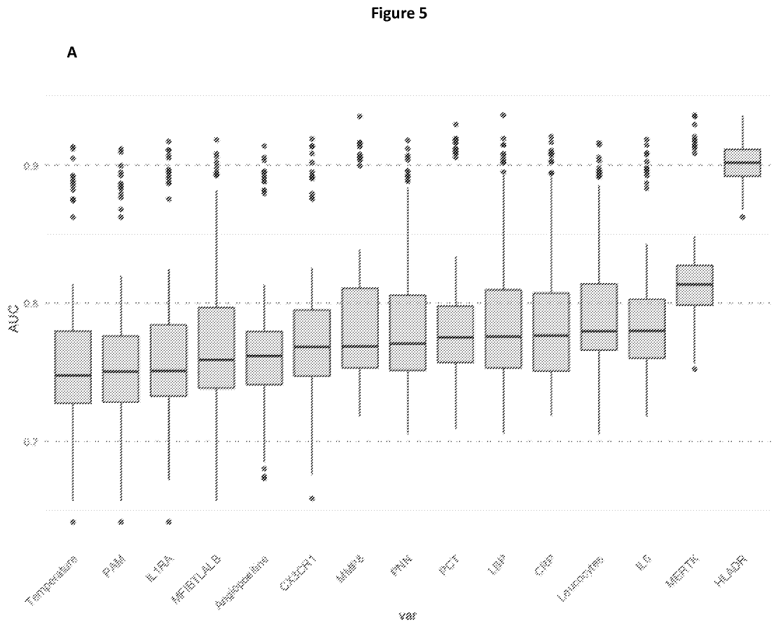

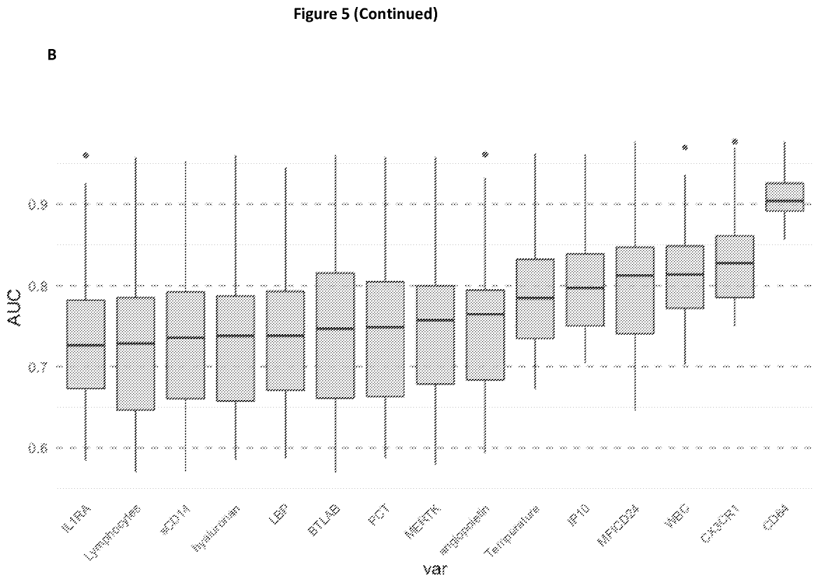

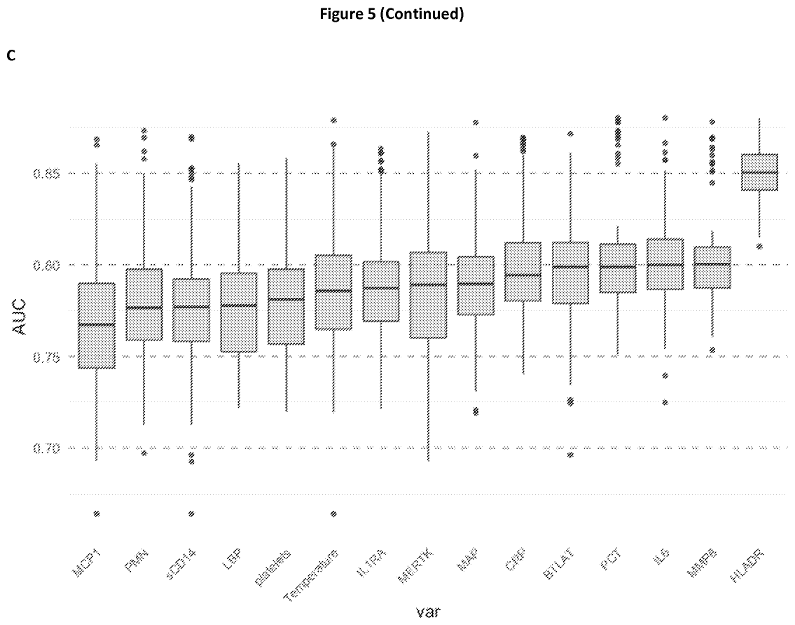

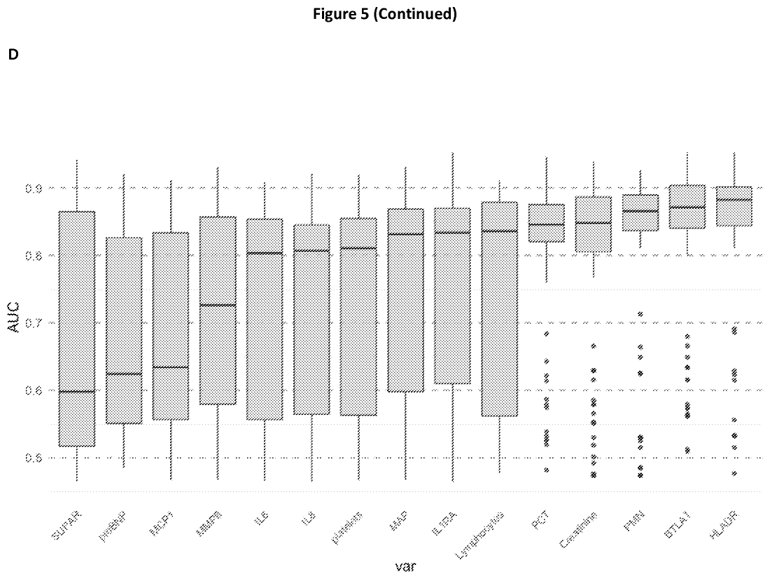

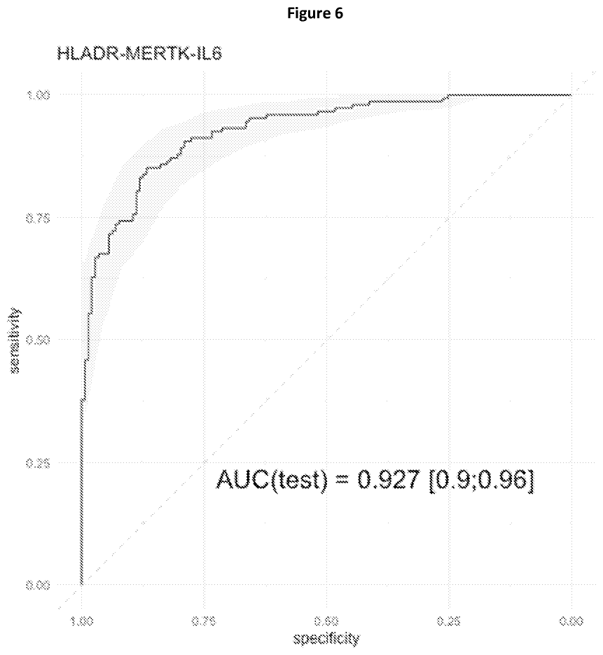

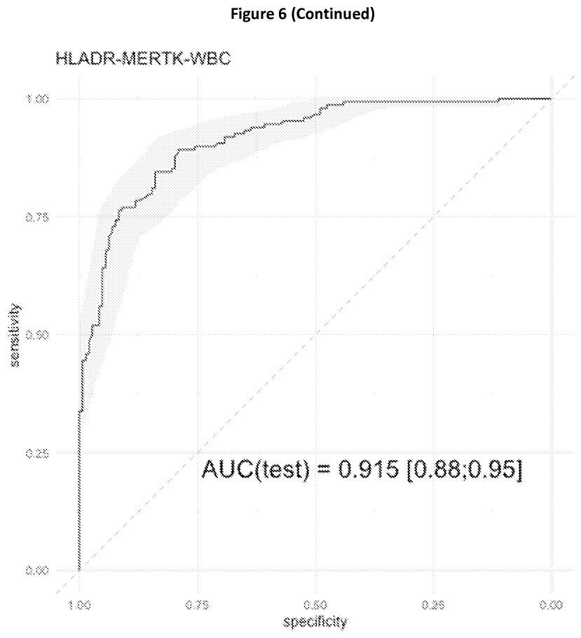

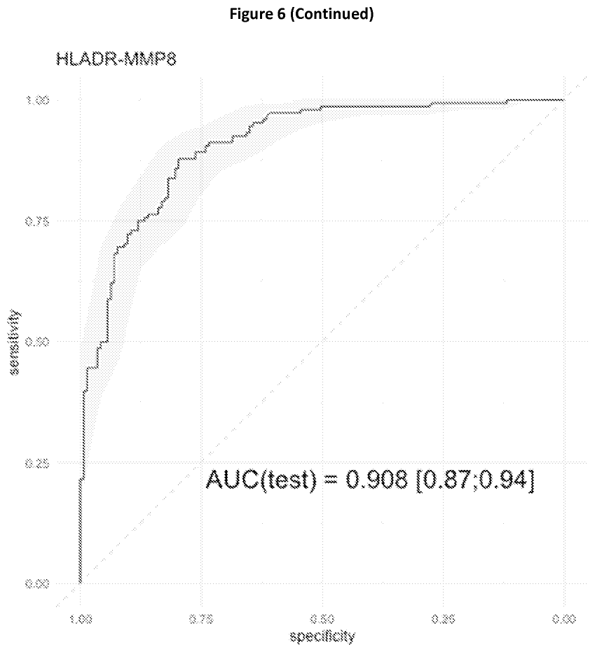

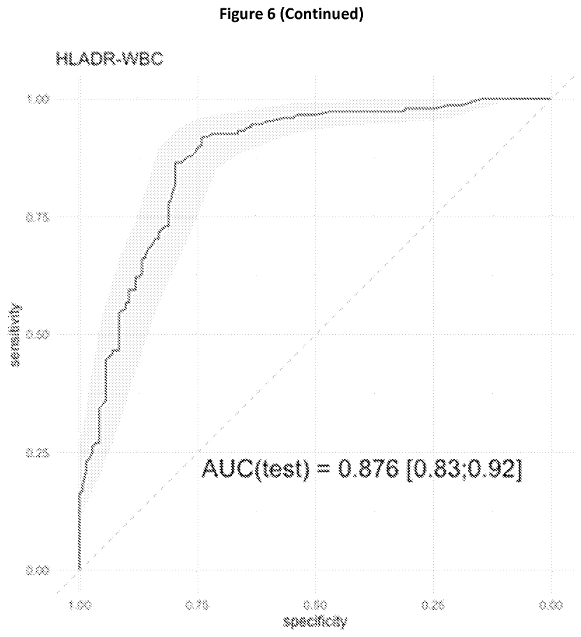

Biomarker's values distribution according to the group of adjudication (bacterial infection, viral infection, no infection, sepsis-2, sepsis-3) are represented on FIG. 2. The statistical combinatory approach identified the association of HLA-DR on monocytes (defined as CD14+ cells), and of MerTk (Myeloid-epithelial reproductive tyrosine kinase) on neutrophils (defined as CD66+ cells) and plasma metalloproteinase-8 as the best combination for bacterial infection with an AUC of 0.94 [95% confidence interval (IC95): 0.91;0.97] (FIG. 3A). The respective strength of each biomarker in the combinatory approach is represented on FIG. 5. Of note, the association of both HLA-DR and MerTK ended with an AUC=0.91 [0.88;0.94]. The addition of a third biomarker improved only moderately the performance (FIG. 6).

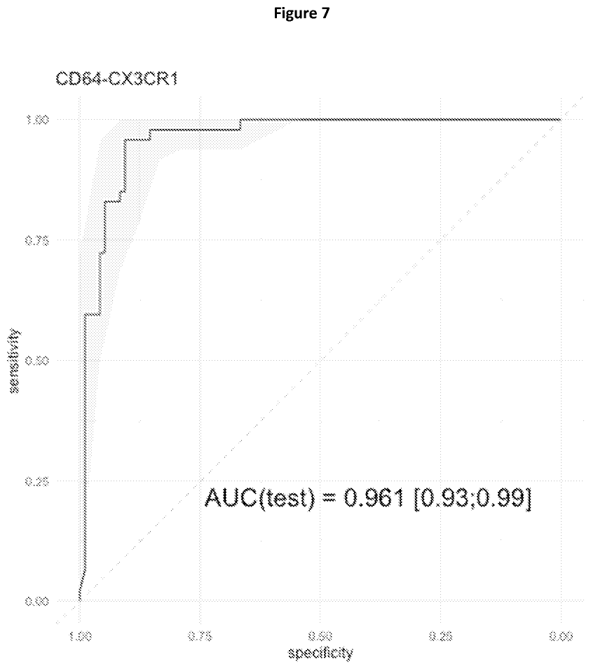

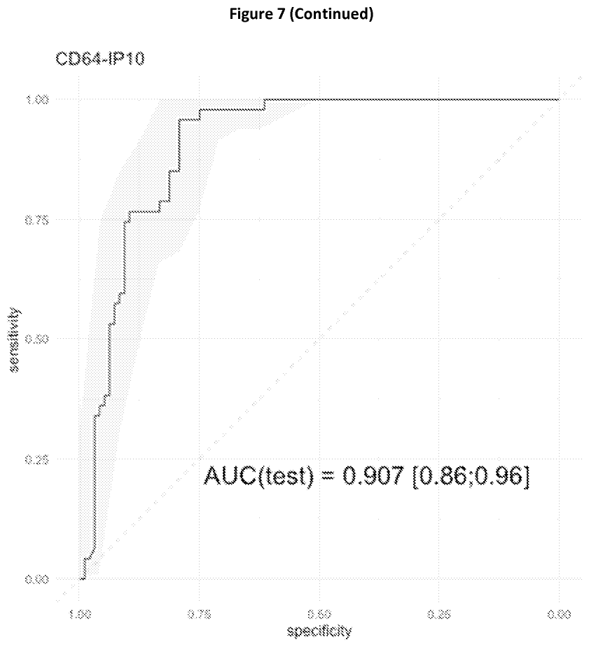

Among the patients who had been adjudicated not to be affected by a bacterial infection with the first biomarker combination, it was possible to define those with a viral infection by the combination of CD64 expression (%), CD24 (MFI) on neutrophils (among CD66+ cells) and CX3CR1 (%) on monocytes (defined as CD14+ cells): AUC=0.98 (0.96;1) (FIG. 2A and 3B). Of note, the AUC of CD64 and CX3CR1 was already 0.96 (0.93;0.99) (FIG. 7).

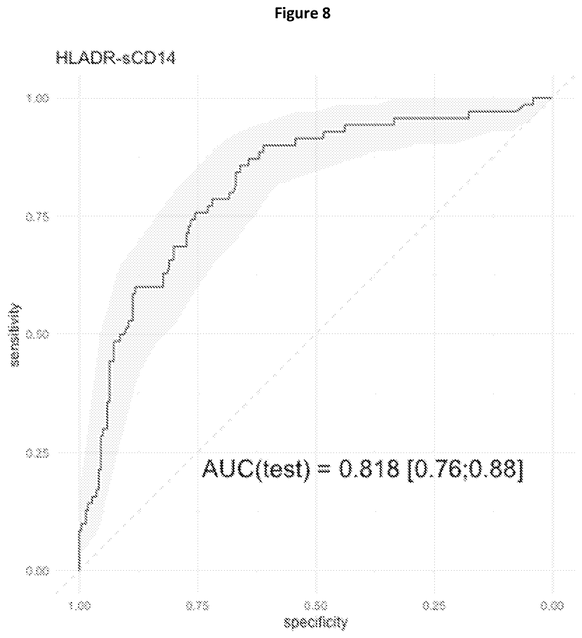

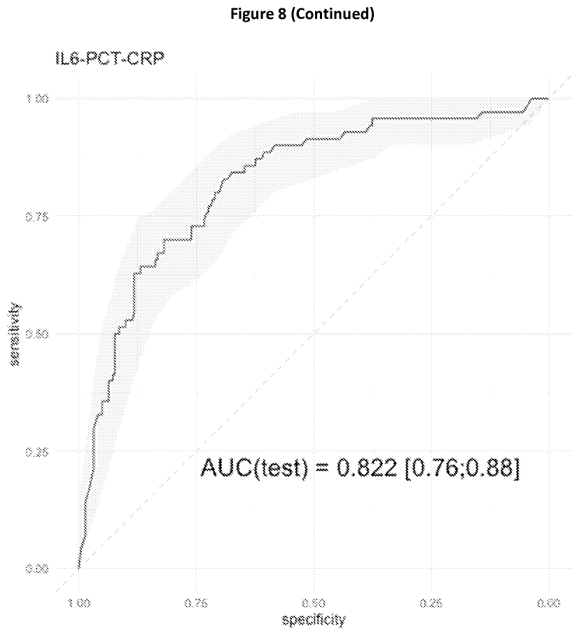

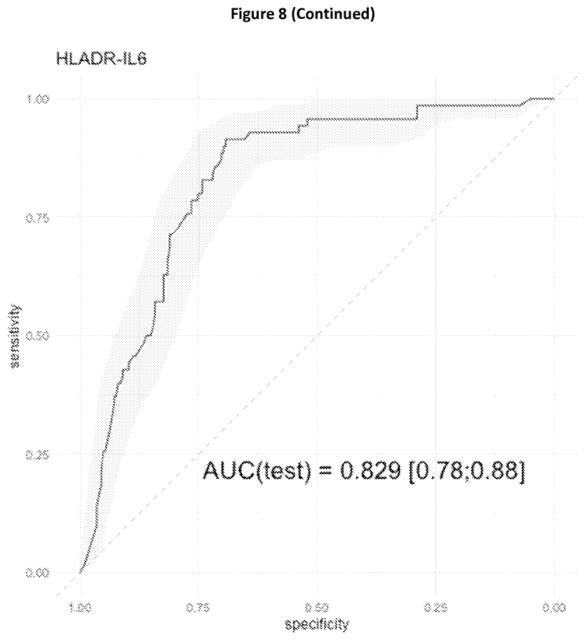

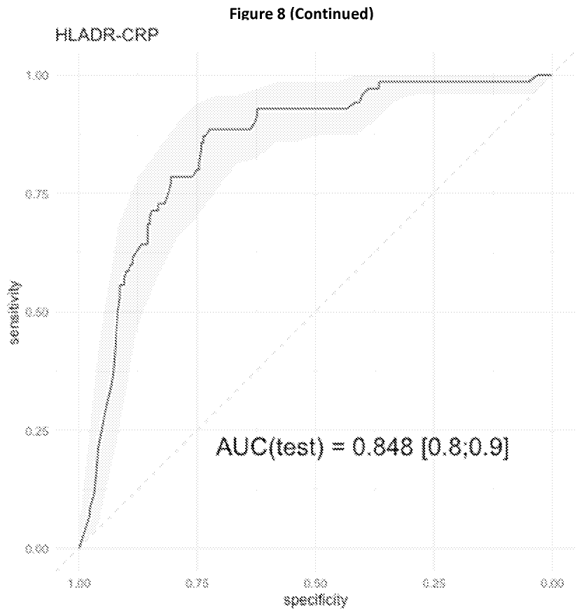

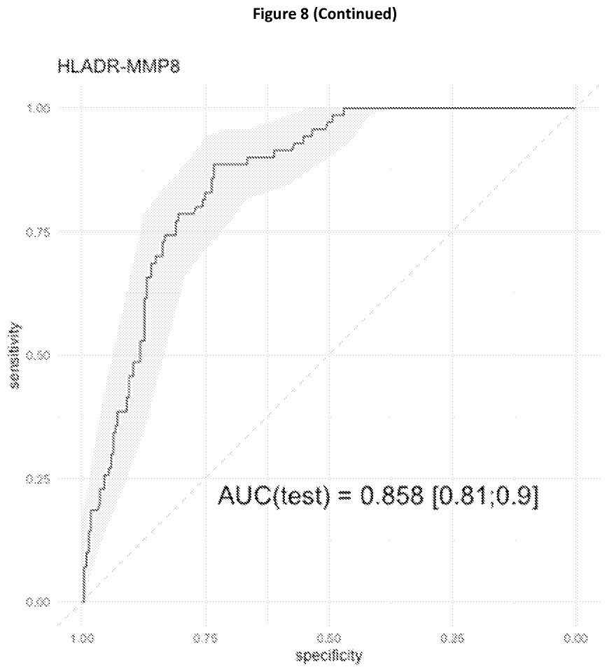

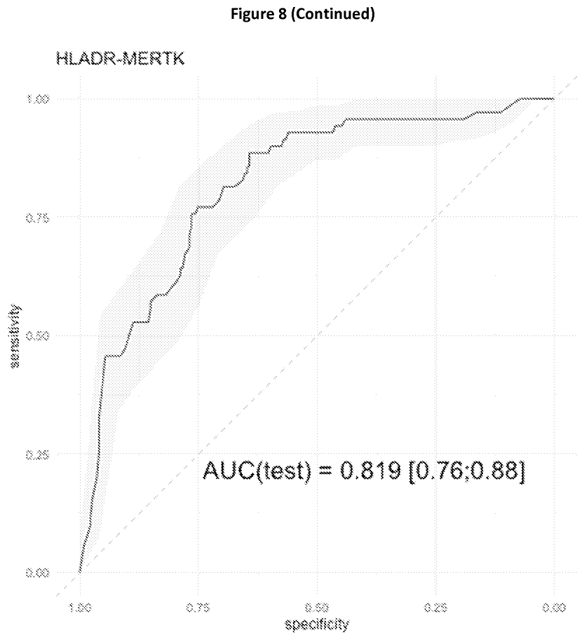

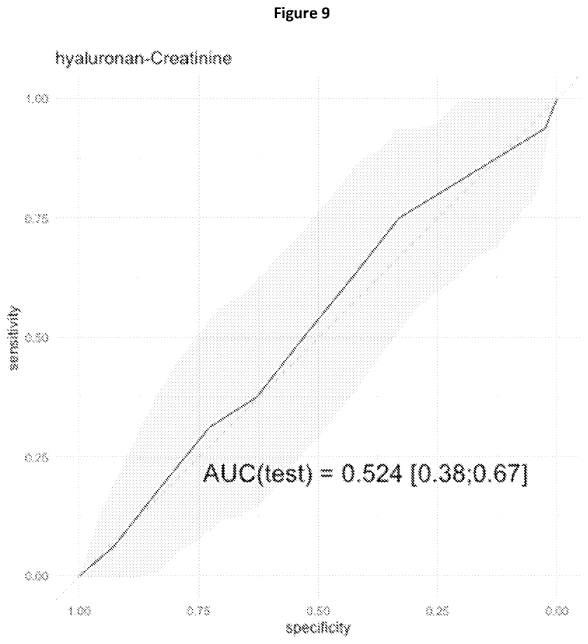

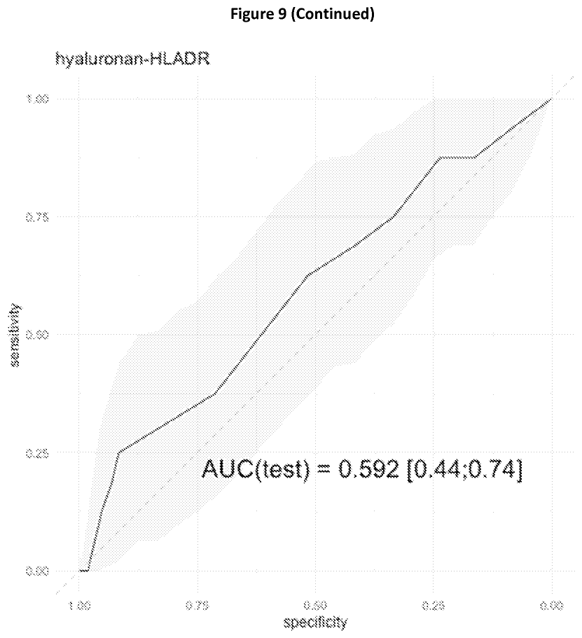

The best combination to define patients with sepsis-2 was HLA-DR, PCT and IL-6 (AUC=0.89 [0.85;0.93]) while the best combination to define patients with sepsis-3 was HLA-DR, hyalurononan and creatinine: AUC=0.92 (0.87;0.97) (FIG. 2B and 2C, 3C and 3D). Other possible combinations are given in FIGS. 8 and 9.

The statistical performances of the best combinations are represented on Table 3.

TABLE 3
statistical performances of different combinations of
biomarkers according to the population of interest.
PPV: positive predictive value. NPV: negative predictive value.
LR+: positive likelihood ratio. LR−: negative likelihood ratio.

| Bacterial | HLADR- | HLADR- | HLADR- | HLADR- | | |
|---|---|---|---|---|---|---|
| infection | MERTK- | MERTK- | MERTK- | MERTK- | HLADR- | HLADR- |
| N = 148 | MMP8 | CX3CR1 | IL6 | WBC | WBC | MMP8 |
| Optimal | 0.434 | 0.356 | 0.477 | 0.593 | 0.393 | 0.328 |
| cut-off | | | | | | |
| Sensitivity | 0.88 | 0.89 | 0.86 | 0.80 | 0.84 | 0.90 |
| | (0.81-0.93) | (0.82-0.93) | (0.79-0.91) | (0.72-0.86) | (0.77-0.89) | (0.84-0.94) |
| Specificity | 0.85 | 0.81 | 0.85 | 0.89 | 0.76 | 0.76 |
| | (0.78-0.90) | (0.74-0.87) | (0.78-0.90) | (0.83-0.93) | (0.69-0.83) | (0.69-0.83) |
| PPV | 0.85 | 0.82 | 0.85 | 0.88 | 0.77 | 0.79 |
| | (0.78-0.90) | (0.75-0.88) | (0.78-0.90) | (0.81-0.93) | (0.70-0.84) | (0.71-0.85) |
| NPV | 0.88 | 0.88 | 0.86 | 0.82 | 0.83 | 0.89 |
| | (0.81-0.93) | (0.81-0.93) | (0.79-0.91) | (0.75-0.87) | (0.76-0.89) | (0.82-0.94) |
| LR+ | 5.93 | 4.69 | 5.79 | 7.37 | 3.55 | 3.82 |
| | (4.01-8.76) | (3.35-6.59) | (3.91-8.56) | (4.61-11.80) | (2.63-4.78) | (2.84-5.12) |
| LR− | 0.14 | 0.14 | 0.16 | 0.23 | 0.21 | 0.13 |
| | (0.09-0.22) | (0.09-0.22) | (0.11-0.25) | (0.16-0.32) | (0.14-0.3) | (0.08-0.21) |
| Viral | CD64- | | | | | |
| infection | CX3CR1- | CD64- | | | | |
| n = 47 | CD24 | CX3CR1 | CD64-IP10 | | | |
| Optimal | 0.644 | 0.713 | 0.716 | | | |
| cut-off | | | | | | |
| Sensitivity | 0.86 | 0.76 | 0.78 | | | |
| | (0.79-0.93) | (0.66-0.84) | (0.68-0.86) | | | |
| Specificity | 0.89 | 0.96 | 0.98 | | | |
| | (0.76-0.96) | (0.84-0.99) | (0.87-1.00) | | | |
| PPV | 0.94 | 0.97 | 0.99 | | | |
| | (0.87-0.98) | (0.90-0.99) | (0.92-1.00) | | | |
| NPV | 0.78 | 0.66 | 0.69 | | | |
| | (0.64-0.88) | (0.54-0.77) | (0.56-0.79) | | | |
| LR+ | 8.22 | 17.87 | 36.72 | | | |
| | (3.58-18.90) | (4.58-69.68) | (5.27-256) | | | |
| LR− | 0.14 | 0.25 | 0.22 | | | |
| | (0.08-0.24) | (0.17-0.36) | (0.15-0.33) | | | |
| Sepsis-2 | HLADR- | IL6-PCT- | HLADR- | HLADR- | HLADR- | HLADR- |
| n = 70 | PCT-IL6 | CRP | PCT | IL6 | CRP | MERTK |
| Optimal | 0.779 | 0.761 | 0.803 | 0.769 | 0.765 | 0.814 |
| cut-off | | | | | | |
| Sensitivity | 0.76 | 0.76 | 0.73 | 0.74 | 0.75 | 0.71 |
| | (0.70-0.81) | (0.69-0.8) | (0.66-0.79) | (0.67-0.79) | (0.68-0.80) | (0.65-0.77) |
| Specificity | 0.87 | 0.77 | 0.86 | 0.89 | 0.80 | 0.84 |
| | (0.77-0.94) | (0.65-0.86) | (0.75-0.93) | (0.78-0.95) | (0.68-0.88) | (0.73-0.92) |
| PPV | 0.95 | 0.91 | 0.94 | 0.95 | 0.92 | 0.94 |
| | (0.90-0.98) | (0.86-0.95) | (0.89-0.97) | (0.91-0.98) | (0.87-0.96) | (0.88-0.97) |
| NPV | 0.54 | 0.50 | 0.50 | 0.52 | 0.50 | 0.48 |
| | (0.44-0.63) | (0.41-0.59) | (0.41-0.59) | (0.42-0.61) | (0.41-0.59) | (0.39-0.57) |
| LR+ | 5.91 | 3.31 | 5.10 | 6.45 | 3.73 | 4.52 |
| | (3.20-10.93) | (2.14-5.12) | (2.86-9.10) | (3.35-12.45) | (2.32-6.00) | (2.61-7.83) |
| LR− | 0.28 | 0.32 | 0.32 | 0.30 | 0.32 | 0.34 |
| | (0.21-0.35) | (0.24-0.41) | (0.25-0.401) | (0.23-0.38) | (0.25-0.41) | (0.27-0.43) |
| | hyaluronan- | | | | | |
| Sepsis-3 | Creatinine- | hyaluronan- | hyaluronan- | | | |
| n = 16 | HLADR | Creatinine | HLADR | | | |
| Optimal | 0.953 | 0.969 | 0.949 | | | |
| cut-off | | | | | | |
| Sensitivity | 0.81 | 0.73 | 0.10 | | | |
| | (0.76-0.85) | (0.67-0.78) | (0.07-0.14) | | | |
| Specificity | 0.94 | 0.94 | 0.94 | | | |
| | (0.68-1.00) | (0.68-1.00) | (0.68-1.00) | | | |
| PPV | 1.00 | 1.00 | 0.96 | | | |
| | (0.97-1.00) | (0.97-1.00) | (0.80-1.00) | | | |
| NPV | 0.22 | 0.17 | 0.06 | | | |
| | (0.13-0.34) | (0.10-0.27) | (0.03-0.09) | | | |
| LR+ | 12.92 | 11.70 | 1.57 | | | |
| | (1.94-86.24) | (1.75-78.12) | (0.23-10.84) | | | |
| LR− | 0.21 | 0.29 | 0.96 | | | |
| | (0.16-0.27) | (0.23-0.36) | (0.84-1.10) | | | |

### Discussion

Using a limited number of biomarkers, it is reported here a very promising approach for the diagnosis of bacterial infection among ED patients. The association of HLA-DR (% on CD14+ monocytes) and MerTK (% on CD66+ neutrophils) resulted in an AUC=0.92 [0.89;0.95] [17,28]. The addition of a third biomarker (MMP8) further improved the AUC at 0.94 [0.91;0.97] with a sensitivity of 0.88 and a specificity of 0.85 (Table 3) [10,29]. Despite the fact that a large number of biomarkers have already been reported to be associated with the diagnosis of infection or sepsis, none had sufficient specificity or sensitivity to be routinely used in clinical practice in ED [4]. When all authors unanimously concluded that there was no “magic maker”, a combination of biomarkers appeared promising [2,30]. For example, the association of CRP and neutrophil CD64, or the association of PCT, soluble TREM-1 and neutrophil CD64 were shown to be promising [31,32]. However, a large multicentre study including 29 plasma biomarkers, 14 cell surface biomarkers and 10 mRNA failed to find any combination useful for the diagnosis of sepsis-2 among ICU patients [33]. Similarly, Lvovschi et al. failed to identify specific cytokine profiles in ED's patients suspected of sepsis [34]. In addition, a recent study analysing numerous cell surface biomarkers concluded that no combination had clinically relevant predictive value for the diagnosis of sepsis among patients with suspected acute infection [35]. This apparent discrepancy with the present results may be explained by the population studied (ED's patients suspected of acute infection, compared to no infection but also to ICU patients with sepsis), the adjudication according to sepsis-3 criteria only, and the choice of logistic regression instead of gradient boosting tree analysis.

It should be noted that most studies carried out for the diagnosis of sepsis have been performed in ICU, in patients with high inflammatory states. For these patients, the occurrence of infection could be hidden within a storm of inflammatory biomarkers and highly altered expression of cell-surface markers.

Implementing sepsis-3 definition in ED is questionable because it only identifies a small proportion of infected patients (roughly those who were classified into severe sepsis in sepsis-2) and by definition those with ongoing organ failure, who are usually already flagged by ED physicians [36-38]. Much more challenging is the accurate identification of patients with bacterial infection which is fundamental for sepsis screening and for improving antibiotic stewardship. Interestingly, among the patients diagnosed in the non-bacterial infection group, thanks to the first combination of biomarkers, it was possible to identify those who had a viral infection. This was achieved with the association of CD64 and CX3CR1 (AUC=0.95 [0.92;0.99]). The addition of a third biomarker on neutrophil (MFI of CD24) [40] produced an AUC of 0.98 [0.96;1]. The sensitivity of these three biomarkers was 0.86 and its specificity 0.89 (Table 3). Very few studies have reported combinations that allow accurate discrimination between bacterial and viral infection. Oved and al. have reported the measurement of three plasma markers (CRP, TRAIL, and CXCL10) and Shapiro et al. recently reported a high accuracy of the combination of point of care CRP and myxovirus resistance protein A measurement, to differentiate bacterial from viral acute upper respiratory infections [42]. Surprisingly, IFN-alpha did not emerge as a biomarker of interest following the statistical combinatory approach in the present study, although it had been previously reported in case of viral infection [26,43].

Since the present study was planned before the Sepsis-3 definition became published, it was designed to find the best combination to identify sepsis-2 patients. In the study, seventy patients were included according to the sepsis-2 definition. The best predictive combination included some well-known biomarkers of sepsis, HLA-DR, PCT and IL-6 [13,47] Regarding the classification of patients according to the Sepsis-3 definition (n=16), the best combination was HLA-DR together with hyaluronan and creatinine [48,49].

### Conclusion

So far, this study is one of the first study published that allows the diagnosis of bacterial infection and of the diagnosis of viral infection in ED′ patients. Because new technologies are rapidly developing, such as the measurement of the expression of cell surface biomarkers by microfluidic and needle shaped microelectrode for electrochemical detection of biomarker in real time, a combination of cell surface biomarkers and plasma biomarkers should not face technical barriers to achieve diagnosis at the bedside [50,51].

### REFERENCES

1. Shankar-Hari M, Phillips G S, Levy M L, Seymour C W, Liu V X, Deutschman C S, et al. Developing a New Definition and Assessing New Clinical Criteria for Septic Shock: For the Third International Consensus Definitions for Sepsis and Septic Shock (Sepsis-3). JAMA. 2016; 315:775
2. Parlato M, Cavaillon J-M. Host response biomarkers in the diagnosis of sepsis: a general overview. Methods Mol Biol. 2015; 1237: 149-211.
3. Levy M M, Artigas A, Phillips G S, Rhodes A, Beale R, Osborn T, et al. Outcomes of the Surviving Sepsis Campaign in intensive care units in the USA and Europe: a prospective cohort study. The Lancet Infectious Diseases. 2012; 12: 919-24
4. Pierrakos C, Vincent J-L, others. Sepsis biomarkers: a review. Crit Care. 2010; 14: R15
5. Turoff M. The design of a policy Delphi. Technological Forecasting and Social Change. 1970; 2:149-71.
6. Hasson F, Keeney S, McKenna H. Research guidelines for the Delphi survey technique. J Adv Nurs. 2000; 32:1008-15.
7. Levy M M, Fink M P, Marshall J C, Abraham E, Angus D, Cook D, et al. 2001 SCCM/ESICM/ACCP/ATS/SIS International Sepsis Definitions Conference. Intensive Care Med. 2003; 29: 530-8.
8. Şirinoğlu M, Soysal A, Karaaslan A, Kepenekli Kadayifci E, Yalindağ-Öztürk N, Cinel I, et al. The diagnostic value of soluble urokinase plasminogen activator receptor (suPAR) compared to C-reactive protein (CRP) and procalcitonin (PCT) in children with systemic inflammatory response syndrome (SIRS). Journal of Infection and Chemotherapy. 2017; 23:17-22.
9. Yang Y, Xie J, Guo F, Longhini F, Gao Z, Huang Y, et al. Combination of C-reactive protein, procalcitonin and sepsis-related organ failure score for the diagnosis of sepsis in critical patients. Ann Intensive Care. 2016 Dec;6(1):51
10. Solan P D, Dunsmore K E, Denenberg A G, Odoms K, Zingarelli B, Wong H R. A novel role for matrix metalloproteinase-8 in sepsis*: Crit Care Med. 2012; 40:379-87.
11. Kofoed K, Andersen O, Kronborg G, Tvede M, Petersen J, Eugen-Olsen J, et al. Use of plasma C-reactive protein, procalcitonin, neutrophils, macrophage migration inhibitory factor, soluble urokinase-type plasminogen activator receptor, and soluble triggering receptor expressed on myeloid cells-1 in combination to diagnose infections: a prospective study. Crit Care. 2007; 11: R38.
12. Gille-Johnson P, Hansson K E, Gårdlund B. Clinical and laboratory variables identifying bacterial infection and bacteraemia in the emergency department. Scand J Infect Dis. 2012; 44:745-52.
13. Zawada et al., SuperSAGE evidence for CD14++CD16+ monocytes as a third monocyte subset. Blood 118(12):e50-61, 2011
14. Hack C E, Hart M, van Schijndel R J, Eerenberg A J, Nuijens J H, Thijs L G, et al. Interleukin-8 in sepsis: relation to shock and inflammatory mediators. Infect Immun. 1992; 60:2835-42.
15. Juillien G, Haroche J, Bonnet P, Rozenberg F, Riou B, Hausfater P. Does serum alpha interferon measurement aid in the etiological diagnosis of febrile adult patients? J Med Virol. 2007; 79:935-8.
16. Ziegler-Heitbrock et al., Nomenclature of monocytes and dendritic cells in blood. Blood, 116(16): e74-80, 2010
17. Wong et al., Gene expression profiling reveals the defining features of the classical, intermediate, and nonclassical human monocyte subsets Blood, 118(5): e16-31, 2011
18. Parlato M, Souza-Fonseca-Guimaraes F, Philippart F, Misset B, Adib-Conquy M, Cavaillon J. CD24-mediated neutrophil death in inflammation: ex vivo study suggesting a potential role in sepsis. Crit Care. 2012; 16: P81.
19. Wang X, Li Z-Y, Zeng L, Zhang A-Q, Pan W, Gu W, et al. Neutrophil CD64 expression as a diagnostic marker for sepsis in adult patients: a meta-analysis. Crit Care. 2015 Jun 10;19(1):245
20. Gámez-Díaz L Y, Enriquez L E, Matute J D, Velásquez S, Gómez I D, Toro F, et al. Diagnostic accuracy of HMGB-1, sTREM-1, and CD64 as markers of sepsis in patients recently admitted to the emergency department. Acad Emerg Med. 2011; 18:807-15.
21. Fleit et al., Monoclonal antibodies to human neutrophil Fc gamma RIII (CD16) identify polypeptide epitopes. Clin Immunol Immunopathol., 59(2): 222-235,1991
22. Shao R, Li C-S, Fang Y, Zhao L, Hang C. Low B and T lymphocyte attenuator expression on CD4+ T cells in the early stage of sepsis is associated with the severity and mortality of septic patients: a prospective cohort study. Crit Care. 2015 Aug 28;19(1):308.
23. Shao R, Fang Y, Yu H, Zhao L, Jiang Z, Li C-S. Monocyte programmed death ligand-1 expression after 3-4 days of sepsis is associated with risk stratification and mortality in septic patients: a prospective cohort study. Crit Care. 2016 May 9;20(1):124.
24. Zhao Y, Jia Y, Li C, Fang Y, Shao R. The risk stratification and prognostic evaluation of soluble programmed death-1 on patients with sepsis in emergency department. Am J Emerg Med. 2018; 36:43-8.
25. Fang Y, Li C, Shao R, Yu H, Zhang Q, Zhao L. Prognostic significance of the angiopoietin-2/angiopoietin-1 and angiopoietin-1/Tie-2 ratios for early sepsis in an emergency department. Crit Care. 2015 Oct 14; 19:367.
26. Rodero M P, Decalf J, Bondet V, Hunt D, Rice G I, Werneke S, et al. Detection of interferon alpha protein reveals differential levels and cellular sources in disease. J Exp Med. 2017; 214:1547-55.
27. Natekin A, Knoll A. Gradient boosting machines, a tutorial. Frontiers in Neurorobotics. 2013; 7:21.
28. Cai B, Kasikara C, Doran A C, Ramakrishnan R, Birge R B, Tabas I. MerTK signaling in macrophages promotes the synthesis of inflammation resolution mediators by suppressing CaMKII activity. Sci Signal. 2018; 11:eaar3721.
29. Fleit et al., A common epitope is recognized by monoclonal antibodies prepared against purified human neutrophil Fc gamma RIII (CD16). Clin Immunol Immunopathol.,62(1 Pt 1): 16-24, 1992
30. Fleuren L M, Klausch T L T, Zwager C L, Schoonmade L J, Guo T, Roggeveen L F, et al. Machine learning for the prediction of sepsis: a systematic review and meta-analysis of diagnostic test accuracy. Intensive Care Med. 2020 Mar;46(3):383-400.
31. Dimoula A, Pradier O, Kassengera Z, Dalcomune D, Turkan H, Vincent J-L. Serial determinations of neutrophil CD64 expression for the diagnosis and monitoring of sepsis in critically ill patients. Clin Infect Dis. 2014;58:820-9.
32. Gibot S, Béné M C, Noel R, Massin F, Guy J, Cravoisy A, et al. Combination Biomarkers to Diagnose Sepsis in the Critically III Patient. Am J Respir Crit Care Med. 2012; 186:65-71.
33. The Captain Study Group, Parlato M, Philippart F, Rouquette A, Moucadel V, Puchois V, et al. Circulating biomarkers may be unable to detect infection at the early phase of sepsis in ICU patients: the CAPTAIN prospective multicenter cohort study. Intensive Care Medicine. 2018; 44:1061-70.
34. Lvovschi V, Arnaud L, Parizot C, Freund Y, Juillien G, Ghillani-Dalbin P, et al. Cytokine profiles in sepsis have limited relevance for stratifying patients in the emergency department: a prospective observational study. PLoS ONE. 2011; 6:e28870.
35. Shankar-Hari M, Datta D, Wilson J, Assi V, Stephen J, Weir CJ, et al. Early PREdiction of sepsis using leukocyte surface biomarkers: the ExPRES-sepsis cohort study. Intensive Care Medicine. 2018; 44:1836-48.
36. Anand V, Zhang Z, Kadri S S, Klompas M, Rhee C. Epidemiology of Quick Sequential Organ Failure Assessment Criteria in Undifferentiated Patients and Association With Suspected Infection and Sepsis. Chest. 2019; 156:289-97.
37. Kalil A C, Machado F R. Quick Sequential Organ Failure Assessment Is Not Good for Ruling Sepsis In or Out. Chest. 2019; 156:197-9.
38. Vincent J-L, Martin G S, Levy M M. qSOFA does not replace SIRS in the definition of sepsis. Crit Care. 2016 Jul 17;20(1):210
39. Ortillon M. et al, Monocyte CD169 expression in COVID-19 patients upon intensive care unit admission. Cytometry A. 2021 May;99(5):466-471.
40. Tamm A. et al., The binding epitopes of human CD16 (Fc gamma RIII) monoclonal antibodies. Implications for ligand binding J Immunol., 157(4): 1576-1581,1996
41. Oved K, Cohen A, Boico O, Navon R, Friedman T, Etshtein L, et al. A Novel Host-Proteome Signature for Distinguishing between Acute Bacterial and Viral Infections. Schildgen O, editor. PLOS ONE. 2015; 10:e0120012.
42. Shapiro N I, Self W H, Rosen J, Sharp S C, Filbin M R, Hou P C, et al. A prospective, multicentre US clinical trial to determine accuracy of FebriDx point-of-care testing for acute upper respiratory infections with and without a confirmed fever. Annals of Medicine. 2018; 50:420-9.
43. Hausfater P, Fillet A-M, Rozenberg F, Arthaud M, Trystram D, Huraux J-M, et al. Prevalence of viral infection markers by polymerase chain reaction amplification and interferon-alpha measurements among patients undergoing lumbar puncture in an emergency department. J Med Virol. 2004; 73:137-46.
44. Sweeney T E, Wong H R, Khatri P. Robust classification of bacterial and viral infections via integrated host gene expression diagnostics. Sci Transl Med. 2016 Jul 6;8(346):346ra91
45. Sweeney T E, Azad T D, Donato M, Haynes W A, Perumal T M, Henao R, et al. Unsupervised Analysis of Transcriptomics in Bacterial Sepsis Across Multiple Datasets Reveals Three Robust Clusters: Critical Care Medicine. 2018; 46:915-25.
46. Miller R R, Lopansri B K, Burke J P, Levy M, Opal S, Rothman R E, et al. Validation of a Host Response Assay, SeptiCyte LAB, for Discriminating Sepsis from Systemic Inflammatory Response Syndrome in the ICU. American Journal of Respiratory and Critical Care Medicine. 2018; 198:903-13.
47. Bourgoin P, Hayman J, Rimmelé T, Venet F, Malergue F, Monneret G. A novel one-step extracellular staining for flow cytometry: Proof-of-concept on sepsis-related biomarkers. J Immunol Methods. 2019 Jul;470:59-63.
48. Bourgoin P, Lediagon G, Arnoux I, Bernot D, Morange P E, Michelet P, Malergue F, Markarian T. Flow cytometry evaluation of infection-related biomarkers in febrile subjects in the emergency department. Future Microbiol. 2020 Feb:15:189-201.
49. Rhee C, et al. Diagnosing sepsis is subjective and highly variable: a survey of intensivists using case vignettes. Crit Care. 2016 Apr 6;20:89.
50. Russell C, Ward A C, Vezza V, Hoskisson P, Alcorn D, Steenson D P, et al. Development of a needle shaped microelectrode for electrochemical detection of the sepsis biomarker interleukin-6 (IL-6) in real time. Biosens Bioelectron. 2019; 126:806-14.
51. Hassan U, Ghonge T, Reddy B, Patel M, Rappleye M, Taneja I, et al. A point-of-care microfluidic biochip for quantification of CD64 expression from whole blood for sepsis stratification. Nat Commun. 2017 Jul 3;8:15949.
52. Velly L. et al, Optimal combination of early biomarkers for infection and sepsis diagnosis in the emergency department: The BIPS study. J Infect. 2021 Apr;82(4):11-21

1-30. (canceled)

31. An in vitro method for blood analysis, said method comprising:
a) isolating monocytes from a blood sample from a subject,
b) measuring the level of HLA-DR (human leukocyte antigen DR) expression on said monocytes,
c) isolating neutrophils from said blood sample,
d) measuring the level of MerTk (Myeloid-epithelial-reproductive tyrosine kinase) expression on said neutrophils,
e) isolating plasma from said blood sample, and
f) measuring the level of Metalloproteinase-8 (MMP8) in said plasma.

32. The method of claim 31, wherein the subject has levels of these markers that are indicative of a bacterial infection.

33. The method of claim 31, wherein the subject has a bacterial infection if the subject displays a decreased level of HLA-DR expression on the monocytes, an increased level of MerTk expression on the neutrophils, and an increased level of MMP8 in the plasma, as compared to reference levels.

34. The method of claim 33, wherein the subject has a bacterial infection if the subject displays a decreased level of HLA-DR expression on the monocytes, an increased level of MerTk expression on the neutrophils, and an increased level of MMP8 in the plasma, as compared to reference levels, said reference levels having been measured on a subject that does not suffer from viral infection nor from bacterial infection.

35. The method of claim 31, wherein the subject does not have a bacterial infection.

36. The method claim 31, further comprising:
g) measuring the level of CD64 expression on the neutrophils from said blood sample,
h) measuring the level of CD24 expression on the neutrophils from said blood sample, and
i) measuring the level of CX3CR1 expression on the monocytes from said blood sample.

37. The method of claim 36, wherein the subject has levels of these markers indicative of a viral infection.

38. The method of claim 36, wherein the subject has a viral infection if a decreased level of CD64 expression, a decreased level of CD24 expression, and an increased level of CX3CR1 expression are observed as compared to reference levels.

39. An in vitro method for diagnosis of a bacterial infection or a viral infection in a subject in need thereof, said method comprising:
a) measuring the level of HLA-DR (human leukocyte antigen DR) expression on the monocytes present in a blood sample of said subject,
b) measuring the level of MerTk (Myeloid-epithelial-reproductive tyrosine kinase) expression on the neutrophils present in said blood sample,
c) measuring the level of Metalloproteinase-8 (MMP8) in the plasma of said blood sample, and
d) diagnosing that the subject suffers from a bacterial infection.

40. The method of claim 39, wherein said subject is diagnosed of bacterial infection if the subject displays a decreased level of HLA-DR expression on the monocytes, an increased level of MerTk expression on the neutrophils, and an increased level of MMP8 in the plasma, as compared to reference levels.

41. The method of claim 39, further comprising the steps of:
e) measuring the level of CD64 expression on the neutrophils in the blood sample,
f) measuring the level of CD24 expression on the neutrophils in the blood sample,
g) measuring the level of CX3CR1 expression on the monocytes in the blood sample, and
h) diagnosing that the subject suffers from a viral infection.

42. The method of claim 31, wherein said subject is diagnosed of viral infection if the subject displays a decreased level of CD64 expression, a decreased level of CD24 expression, and an increased level of CX3CR1 expression, as compared to reference levels.

43. An in vitro method for selecting a therapy, said method comprising diagnosing a patient with the method according to claim 39 and administering an antimicrobial or antiviral agent to the subject, depending on the results of said diagnostic methods.

44. A method for treating patients, said method comprising:
diagnosing a patient with the method of claim 39,
administering antibacterial agents to patients whose blood samples display a decreased level of HLA-DR (human leukocyte antigen DR) expression on the monocytes, an increased level of MerTk (Myeloid-epithelial-reproductive tyrosine kinase) expression on the neutrophils, and an increased level of Metalloproteinase-8 (MMP8) in the plasma, as compared to reference levels.
